(19) United States
(12) Patent Application Publication
(10) Pub. No.: US 2025/0186407 A1
(43) Pub. Date: Jun. 12, 2025

(54) TRANSMUCOSAL THERAPEUTIC SYSTEM CONTAINING A MACROLIDE IMMUNOSUPPRESSANT

(71) Applicants: LTS LOHMANN THERAPIE-SYSTEME AG, Andernach (DE); NUCLEUS MEDICAL GMBH, Grünwald (DE)

(72) Inventors: Marius BAUER, Andernach (DE); Antje ZILLER, Vallendar (DE); Marlene FUHRMANN, Kail (DE); Frank SEIBERTZ, Bad Breisig (DE)

(21) Appl. No.: 18/844,505

(22) PCT Filed: Mar. 8, 2023
(86) PCT No.: PCT/EP2023/055945

§ 371 Date: Sep. 6, 2024

(30) Foreign Application Priority Data
Mar. 11, 2022 (EP) 22161725.1

(51) Int. Cl. A61K 31/436; A61K 9/70; A61K 47/10; A61K 47/32; A61K 47/38

(52) U.S. Cl. CPC A61K 31/436; A61K 9/7084; A61K 47/10; A61K 47/32; A61K 47/38

(57) ABSTRACT

The present invention relates to transmucosal therapeutic systems for the transmucosal administration of an active agent comprising a mucoadhesive layer structure comprising a macrolide immunosuppressant as active agent, such transmucosal therapeutic systems for use in a method of treatment and/or prophylaxis, and processes of manufacture of such transmucosal therapeutic systems.

### TECHNICAL FIELD OF THE INVENTION

The present invention relates to a transmucosal therapeutic system for the transmucosal administration of a macrolide immunosuppressant to the systemic circulation, and processes of manufacture, methods of treatment and uses thereof.

### BACKGROUND OF THE INVENTION

Macrolides are natural or partially synthetic macrocyclic compounds comprising a large lactone ring. Some of the macrolides present antibiotic or antifungal properties, while others act as immunomodulators, i.e., they are capable of adjusting, e.g., activating or suppressing the immune system. Immunosuppressive drugs are in general used mainly as medicaments in case of organ transplantation and autoimmune diseases to increase immune tolerance and to stop the immune system from attacking its own or transplanted foreign organs.

Tacrolimus, everolimus, sirolimus and pimecrolimus are, e.g., non-antibiotic macrolide immunosuppressants. While the first three are used in organ transplantation medicine to prevent organ rejection, pimecrolimus, but also tacrolimus is used to treat inflammatory skin diseases. The macrolides share a high molecular weight and low solubility in aqueous media.

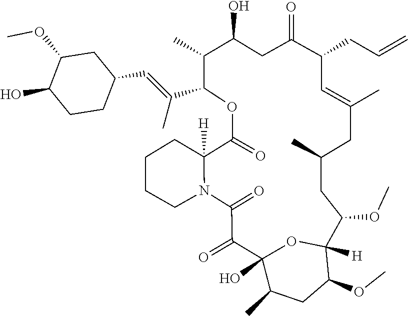 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

### Tacrolimus

Tacrolimus is for example sold under the brand name Prograf® by Astellas Pharma in the form of oral capsules comprising 0.5, 1 or 5 mg tacrolimus, and is indicated for the prophylaxis of organ rejection in patients receiving allogeneic liver, kidney or heart transplant.

Oral dosage forms such as capsules are easy and convenient to take and allow self-administration. However, these types of dosage forms have the disadvantage that the drug is absorbed via the gastrointestinal (GI) tract and that the bioavailability is often greatly reduced due to being metabolized before reaching the systemic circulation (so-called first-pass effect). The bioavailability of tacrolimus in Prograf® is indeed reported to be low, and the drug release appears to vary to some extend due to food effect when taken orally.

The oral mucosa is an administration site that is not yet as much used as traditional oral dosage forms but offers the advantage of combining the ease and convenience of use with bypassing the GI-tract, since the drug is absorbed into the systemic circulation directly via the mucosal tissue.

Transmucosal therapeutic systems, or transmucosal delivery systems (sometimes also termed buccal patches, oromucosal films, etc.), consist of one or more thin layers which are applied and adhere to the mucosa of the oral cavity to deliver the drug over a period of time. Dosage forms in the form of thin films for application in the oral cavity are also sometimes referred to as “Oral Thin Film” or OTF, however, many OTFs are not intended to adhere to the mucosa. In a transmucosal therapeutic system, the active is typically contained in a dissolvable or erodible layer, which often has mucoadhesive properties. As the film adheres to the mucosa, delivery of the active ingredient may be achieved by a combination of direct active release from the transmucosal therapeutic system to the mucosa, and, in the absence of a backing layer, by an indirect delivery of the active via dissolution in the saliva, often followed by swallowing of the saliva and absorption of the active ingredient from the gastrointestinal tract. Ideally, OTFs do not provoke any negative sensation of having a disturbing object in the mouth, since the film, adhered to the mucosa, does not freely move around in the oral cavity, and since the film is usually thin enough not to be perceived by the patient once applied.

However, transmucosal therapeutic systems are a relatively new form of drug delivery, meaning that knowledge on formulation technology is limited. Formulating appropriate dosage forms for the transmucosal delivery by OTFs is challenging due to a multitude of aspects to be considered and issues to be solved.

One such aspect is the aqueous environment of the oral cavity, due to which most of the film-forming polymers used in transmucosal therapeutic systems are water-soluble, and it is often difficult to find a formulation for hydrophobic drugs and/or those with relatively high molecular weight such as macrolide immunosuppressants, which is appropriate for delivering therapeutically effective amounts of the drug and still provides sufficient mucoadhesion. A low solubility in aqueous media does not only mean that the saturation concentration of the drug in the drug layer might be lower than the required drug amount so that the drug risks to precipitate, but also that the drug release is slow due to the limited solubility in the oral aqueous environment. The low solubility affects both the drug release from the matrix as well as the erosion of the matrix layer, which needs to be adapted to the drug hydrophobicity. Otherwise, the matrix would erode too fast and before the drug is absorbed via the mucosa, releasing the drug into the saliva. The indirect delivery via the saliva is unwanted in particular for slowly releasing drugs due to the risk of unintended swallowing and loss to the GI system.

A mucoadhesive buccal film containing nanoparticles of tacrolimus has been proposed recently. However, the use of nanoparticles to increase the solubility of tacrolimus is complex and expensive, since the nanoparticles have to be manufactured in a separate step and a process control to characterize the nanoparticles has to be implemented. Another known tacrolimus buccal film is based on Chitosan and hydroxypropyl methyl cellulose HPMC, but comprises only low amounts of tacrolimus (about 2 wt-% tacrolimus in a 15 mg film, i.e., comprising about 0.3 mg tacrolimus per film).

In summary, a transmucosal therapeutic system of macrolide immunosuppressants such as tacrolimus overcoming the disadvantages of the known systems is much needed. As outlined above, a transmucosal therapeutic system would be able to address the disadvantage of low bioavailability of the oral administration route, but it is difficult to find an appropriate formulation for drugs with low solubility.

Thus, there is a need for a transmucosal therapeutic system of macrolide immunosuppressants such as tacrolimus with improved but simple formulation, ensuring a sufficient and appropriate delivery of the drug.

### OBJECTS AND SUMMARY OF THE INVENTION

It is an object of the present invention to provide a transmucosal therapeutic system for the transmucosal administration of a macrolide immunosuppressant overcoming the above-mentioned disadvantages of current systems.

Thus, it is an object of the present invention to provide a transmucosal therapeutic system for the transmucosal administration of a macrolide immunosuppressant that incorporates a sufficient, e.g., a therapeutically effective amount of the drug.

Thus, it is an object of the present invention to provide a transmucosal therapeutic system for the transmucosal administration of a macrolide immunosuppressant that incorporates a sufficient, e.g., a therapeutically effective amount of the drug.

It is a further object of the present invention to provide a transmucosal therapeutic system for the transmucosal administration of a macrolide immunosuppressant which is also capable of delivering a therapeutically effective amount of the drug to the systemic circulation.

It is another object of the present invention to provide a transmucosal therapeutic system for the transmucosal administration of a macrolide immunosuppressant which provides sufficient mucoadhesion and a unidirectional delivery of the drug through the mucosa.

It is yet another object of the present invention to provide a transmucosal therapeutic system for the transmucosal administration of a macrolide immunosuppressant which is also simple and cost-efficient to manufacture.

These objects and others are accomplished by the present invention, which according to one aspect relates to a transmucosal therapeutic system for the transmucosal administration of an active agent comprising
a mucoadhesive layer structure comprising
A) a backing layer comprising a first film-forming polymer,
B) an adhesive layer comprising
   a laminating adhesive; and
   a first plasticizer, and
C) an active agent-containing layer comprising
   a macrolide immunosuppressant; and
   a second film-forming polymer,
      wherein
the first film-forming polymer is ethyl cellulose,
the macrolide immunosuppressant is selected from the group consisting of tacrolimus, sirolimus, everolimus and pimecrolimus,
and
the second film-forming polymer is selected from the group consisting of ethyl cellulose, hydroxypropyl cellulose, and any mixtures thereof.

According to another aspect, the invention relates to an active agent-containing layer for a transmucosal therapeutic system, the active agent-containing layer comprising
i) tacrolimus in amorphous form; and
ii) a film-forming polymer selected from the group consisting of ethyl cellulose, hydroxypropyl cellulose, and any mixtures thereof.

According to a further aspect, the invention relates to a mucoadhesive layer structure for a transmucosal therapeutic system comprising
A) an active agent-containing layer comprising
   i) tacrolimus in amorphous form; and
   ii) a film-forming polymer selected from the group consisting of ethyl cellulose, hydroxypropyl cellulose, and any mixtures thereof,
B) an adhesive layer comprising
   a laminating adhesive; and
   a plasticizer.

According to yet another aspect, the invention relates to a process of manufacture of a transmucosal therapeutic system comprising the steps of
(a) combining at least
   a first film-forming polymer and
   a solvent
   to obtain a backing layer coating composition,
   coating the backing layer coating composition onto a release liner and
   drying the coated backing layer coating composition to form a backing layer,
(b) combining at least
   an active agent,
   a second film-forming polymer, and
   a solvent
   to obtain an active agent layer coating composition,
   coating the active agent layer coating composition onto a release liner and
   drying the coated active agent layer coating composition to form a single-layer active agent-containing layer, optionally repeating the coating step by coating the active agent layer coating composition on the coated active agent layer coating composition before drying, or on the dried active agent-containing layer to obtain a double-layer active agent-containing layer,
(c) combining at least
   a laminating adhesive, and
   a first plasticizer, and
   a solvent
   to obtain an adhesive layer coating composition,
   coating the adhesive layer coating composition onto the backing layer obtained in step (a), and
   drying the coated adhesive layer coating composition to form a bi-layer laminate consisting of an adhesive layer and a backing layer on the release liner, and
(d) laminating the bi-layer laminate obtained in step (c) with the single- or double-layer active agent-containing layer obtained in step (b),
   wherein
the first film-forming polymer is ethyl cellulose,
the macrolide immunosuppressant is selected from the group consisting of tacrolimus, sirolimus, everolimus and pimecrolimus,
and
the second film-forming polymer is selected from the group consisting of ethyl cellulose, hydroxypropyl cellulose, and any mixtures thereof.

According to an even further aspect of the invention, the transmucosal therapeutic system according to the invention is for use in a method of treatment, preferably for the treatment and/or prophylaxis of organ rejection after organ transplant in a human patient, and more preferably for the prophylaxis of organ rejection after allogeneic liver, kidney or heart transplant, in a human patient.

According to another aspect, the invention relates to a transmucosal therapeutic system for the transmucosal administration of an active agent comprising a mucoadhesive layer structure consisting of
A) a backing layer comprising
   from 55 to 95 wt-% and preferably about 60 wt-% of ethyl cellulose with a viscosity as measured as a 5% solution at 25° C. in 80:20 toluene:ethanol by weight in accordance with ASTM D914 of from 30 to 60 mPa·s, more preferably from 40 to 52 mPa·s, and a substitution of ethoxyl groups of 48.0 to 49.5% as measured in accordance with ASTM D914,
   from 5 to 20 wt-% and preferably about 10 wt-% triacetin, and
   from 10 to 35 wt-% and preferably about 30 wt-% of a mixture consisting of
      about 19 wt-% polyvinylpyrrolidone,
      about 80 wt-% polyvinylacetate,
      about 0.8 wt-% sodium lauryl sulfate and
      about 0.2 wt-% of silica,
B) an adhesive layer adjacent to the active agent-containing layer on its one side and to the backing layer on its other side, comprising
   from 70 to 90 wt-% and preferably about 80 wt-% of a mixture consisting of
      about 19 wt-% polyvinylpyrrolidone,
      about 80 wt-% polyvinylacetate,
      about 0.8 wt-% sodium lauryl sulfate and
      about 0.2 wt-% of silica,
   from 10 to 30 wt-%, preferably about 15% of glycerin, and
   from 0 to 10 wt-%, preferably about 5 wt-% of ethyl cellulose with a viscosity as measured as a 5% solution at 25° C. in 80:20 toluene:ethanol by weight in accordance with ASTM D914 of from 30 to 60 mPa·s, more preferably from 40 to 52 mPa·s, and a substitution of ethoxyl groups of 48.0 to 49.5% as measured in accordance with ASTM D914,
C) an active agent-containing layer comprising
   at least 0.8 mg/cm² of tacrolimus in amorphous form;
      ethyl cellulose with a viscosity as measured as a 5% solution at 25° C. in 80:20 toluene:ethanol by weight in accordance with ASTM D914 of from 30 to 60 mPa·s, more preferably from 40 to 52 mPa·s, and a substitution of ethoxyl groups of 48.0 to 49.5% as measured in accordance with ASTM D914, in an amount of 25 to 35 wt-% of the active agent-containing layer,
      hydroxypropyl cellulose with a molecular weight (as measured by GPC-size exclusion chromatography) of about 370,000, and a Brookfield Viscosity of 150-400 mPa·s as measured as a 2% solution in water, in an amount of 7 to 15 wt-% of the active agent-containing layer, and
      hydroxypropyl cellulose with a molecular weight (as measured by GPC-size exclusion chromatography) of about 80,000, and a Brookfield Viscosity of 300-600 mPa·s as measured as a 10% solution in water, in an amount of 30 to 45 wt-% of the active agent-containing layer,
   a mixed calcium/sodium salt of a methyl vinyl ether and maleic anhydride copolymer as a further film-forming polymer in an amount of 2 to 5 wt-% of the active agent-containing layer,
   optionally a vinylpyrrolidone-vinyl acetate copolymer in an amount of 1 to 4 wt-% of the active agent-containing layer, and
   glycerin in an amount of 3 to 6 wt-% of the active agent-containing layer.

Within the meaning of this invention, the term “transmucosal therapeutic system” or “transmucosal delivery system” refers to a system by which an active agent (e.g. tacrolimus) is administered to the systemic circulation via transmucosal delivery by application to the mucosa of the oral cavity, and refers to the entire individual dosing unit that is applied to the mucosa of a patient, and which comprises a therapeutically effective amount of the active agent in a mucoadhesive layer structure. The mucoadhesive layer structure may be located on a release liner (a detachable protective layer), thus, the transmucosal therapeutic system may further comprise a release liner. In contrast to certain oral thin films which are not necessarily mucoadhesive and which are intended to disintegrate very fast in the saliva (sometimes referred to as “flash wafers”), enteral delivery is unintended and undesirable in transmucosal therapeutic systems.

Within the meaning of this invention, the term “mucoadhesive layer structure” refers to an active agent-containing multi-layer laminate structure providing the area of release for the active agent during administration.

As used herein, the expression “active agent” means a biologically or pharmacologically active compound, which may also be referred to as active, drug substance, drug, active ingredient, active pharmaceutical ingredient (API), or the like. Within the meaning of this invention, the term “therapeutically effective amount” refers to a quantity of active agent in the transmucosal therapeutic system sufficient to provide, if administered by the transmucosal therapeutic system to a patient, the desired therapeutic effect, e.g., as determined by blood levels of a similar range (e.g. of about 10% to about 1000% as measured as an AUC) when compared to blood levels obtained after a one-time administration of approved drug products of the same active agent.

There are two main types of transmucosal therapeutic systems, i.e. those using backing layers, and those without. The delivery of an active ingredient by an open system type transmucosal therapeutic system using no backing layer will always be a combination of direct delivery from the transmucosal therapeutic system through the mucosa at the adhesion site, and indirect delivery via dissolution of the active from the transmucosal therapeutic system into the saliva, and from the saliva through the mucosa of the oral cavity (and also the stomach or the intestines in case the saliva is swallowed). The proportion of the different delivery routes depends, effects of permeability via the mucosa notwithstanding, mainly on factors such as the solubility of the active and the disintegration time of the transmucosal therapeutic system. The higher the solubility and the faster the disintegration of the transmucosal therapeutic system, the more dissolution into the saliva will be favored over direct delivery into the mucosa at the adhesion site. Such an indirect delivery may have the advantage of providing a practical increase of the mucosal surface area through which the active is released systemically. However, dissolution into the saliva means that the active concentration, and thus the final delivered amount is difficult to control, and the risk of enteral delivery by unintended swallowing of the saliva is serious. The present invention therefore is directed to transmucosal therapeutic systems comprising a backing layer.

Transmucosal therapeutic systems using a backing layer have a completely different approach. In such systems, by using a backing layer, the delivery route is substantially limited to the direct transmucosal delivery, which can be much better controlled, the risk of enteral delivery is reduced, and, even more advantageously, by limiting dissolution of the active agent into the saliva, it is believed that any irritating sensation or bad taste potentially caused by an active agent can be substantially reduced. The challenge for transmucosal therapeutic systems using a backing layer lies in nonetheless providing a sufficient transmucosal permeation and delivery of active. Thus, in the sense of the present invention, a “backing layer” is any layer within a transmucosal therapeutic system which is able to prevent (at least a substantial amount of) the active contained within the transmucosal therapeutic system to be dissolved into the saliva. Such a backing layer can be non-dissolvable, or sparingly dissolvable over a prolonged period of time. In the latter case, the time the backing layer takes for dissolution is at least as long as (a substantial amount of) the active takes to be delivered through the mucosa.

The active agent-containing layer may be the final, solidified layer, e.g., obtained after coating and drying the solvent-containing coating composition. The active agent-containing layer may also be manufactured by laminating two or more such solidified layers (e.g., dried layers) of the same composition to provide the desired area weight. The active agent-containing layer may be mucoadhesive (in the form of a mucoadhesive layer) or the transmucosal therapeutic system may comprise an additional mucosa-contacting layer of a mucoadhesive for providing sufficient adhesion. Advantageously, the active agent-containing layer is a mucoadhesive layer.

Within the meaning of this invention, the term “mucoadhesive” refers to a material that in particular adheres to and upon contact with a mucosa, but which preferably is non-tacky and can be touched, e.g., with the fingers and manipulated, e.g., for application into the oral cavity, without unintentionally adhering to the skin of the fingers, when in dry state. A mucoadhesive layer, when in contact with the mucosa, is “self-adhesive”, i.e., provides adhesion to the mucosa so that typically no further aid for fixation is needed. The adhesion strength is preferably strong enough that typical movements in the oral cavity are not sufficient to displace a mucoadhesive layer adhered to the mucosa. A “mucoadhesive” layer structure includes a mucoadhesive layer for mucosa contact which may be provided in the form of a mucoadhesive active agent-containing layer or in the form of an additional layer, i.e., a mucoadhesive mucosa-contacting layer.

Within the meaning of this invention, the term “mucosa-contacting layer” refers to a layer included in the transmucosal therapeutic system to be in direct contact with the mucosa of the patient during administration. If the transmucosal therapeutic systems comprises a mucosa-contacting layer as well as one or more other layers, the other layers do not have to contact the mucosa and do not necessarily have mucoadhesive properties. The area of release is provided by the area of the active agent-containing layer. A mucosa-contacting layer which is not at the same time the active agent-containing layer may be used to enhance adherence. The sizes of an additional mucosa-contacting layer and the active agent-containing layer are usually coextensive and correspond to the area of release.

Within the meaning of this invention, the term “area weight” refers to the dry weight of a specific layer, e.g., of the active agent-containing layer, provided in g/m². The area weight values are subject to a tolerance of ±10%, preferably +7.5% of the nominal value, due to manufacturing variability.

The unit “%” may refer to a percentage given in weight per volume (w/v), volume per volume (v/v) or in weight-%, and, if not indicated otherwise, “%” preferably refers to weight-%.

Within the meaning of this invention, the term “polymer” refers to any substance or material consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2,000, preferably above 5,000 and more preferably above 10,000 Dalton. Correspondingly, compounds with a molecular weight below 2,000, preferably below 5,000 or more preferably below 10,000 Dalton are usually referred to as oligomers.

Within the meaning of this invention, the term “administration” refers to the application of the dosage form, i.e., the transmucosal therapeutic system, to the oral mucosa of the patient, which is then maintained on the mucosa for a specified period of time of administration duration, or until the active agent-containing layer is dissolved, eroded or substantially disintegrated.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

Within the meaning of this invention, the term “coating composition” refers to a composition comprising all components of one of the layers of the mucoadhesive layer structure in a solvent, which may be coated to form the corresponding layer upon drying.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, heptanes, toluene and mixtures thereof.

Within the meaning of this invention, and unless otherwise specified, the term “about” refers to an amount that is +10% of the disclosed amount. In some embodiments, the term “about” refers to an amount that is +5% of the disclosed amount. In some embodiments, the term “about” refers to an amount that is +2% of the disclosed amount.

Within the meaning of this invention, the term “substantial” or “substantially” is used to refer to a large part of the corresponding object or component concerned, e.g., a composition “consisting substantially of” a component comprises a large amount of such a component, such as at least 95% by weight, preferably at least 98% or even at least 99% by weight.

### BRIEF DESCRIPTION OF THE DRAWINGS

FIG. 1 depicts a schematic illustration of a process of manufacture of the transmucosal therapeutic system according to one embodiment.

FIG. 2 depicts a comparison of the XRD diffractograms obtained for Layer E and for Placebo Layer E and the XRPD diffractograms obtained for pure crystalline tacrolimus.

FIG. 3 depicts a comparison of the XRD diffractograms obtained for Layer J and for Placebo Layer J and the XRPD diffractograms obtained for pure crystalline tacrolimus.

FIG. 4 depicts a comparison of the XRD diffractograms obtained for Layer K and for Placebo Layer K and the XRPD diffractograms obtained for pure crystalline tacrolimus.

FIG. 5 depicts a comparison of the XRD diffractograms obtained for Reference Layer I and for Placebo Layer I and the XRPD diffractograms obtained for pure crystalline tacrolimus.

FIG. 6 depicts the cumulative released amount of tacrolimus over time obtained in the in vitro release experiment of Example 4 for transmucosal therapeutic systems prepared according to Examples 2E, 2H and 2I.

FIG. 7 depicts the release rate of tacrolimus over time obtained in the in vitro release experiment of Example 4 for transmucosal therapeutic systems prepared according to Examples 2E, 2H and 2I.

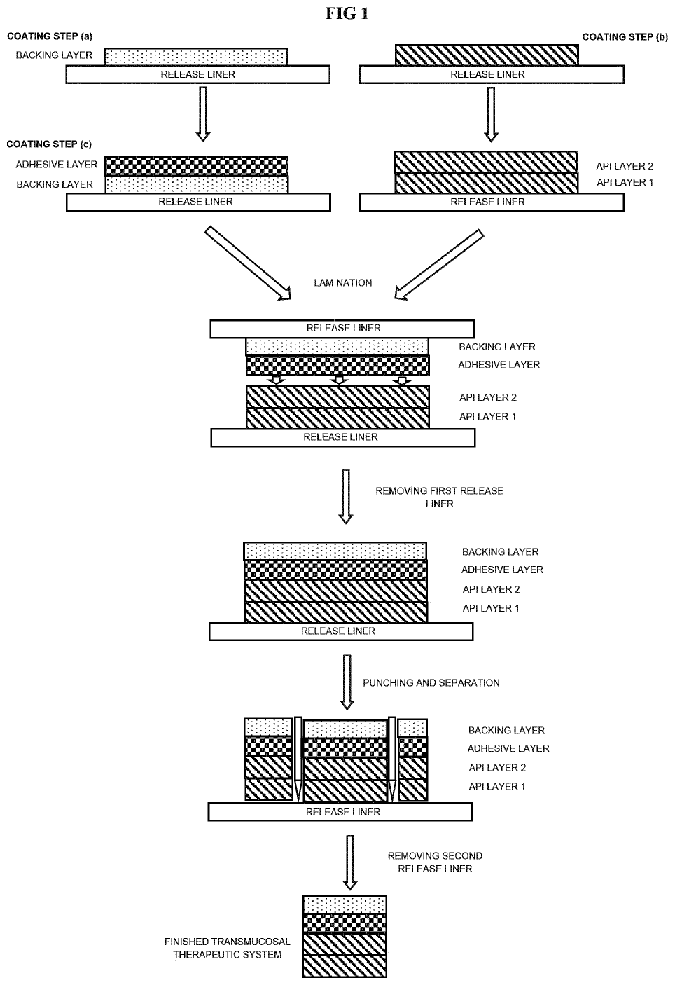

### DETAILED DESCRIPTION

### Structure of the Transmucosal Therapeutic System

The present invention is related to a transmucosal therapeutic system comprising a mucoadhesive layer structure comprising a backing layer, an adhesive layer and an active agent-containing layer.

The transmucosal therapeutic system is intended to be used for the transmucosal administration of an active agent useful in the treatment and/or prophylaxis of diseases and medical conditions. The backing layer comprises a film-forming polymer, the adhesive layer comprises a laminating adhesive and a plasticizer, and the active agent-containing layer comprises a macrolide immunosuppressant and also a film-forming polymer.

Thus, in accordance with the present invention, the transmucosal therapeutic system for the transmucosal administration of an active agent comprises a mucoadhesive layer structure comprising:
A) a backing layer comprising a first film-forming polymer,
B) an adhesive layer comprising
   a laminating adhesive; and
   a first plasticizer, and
C) an active agent-containing layer comprising
   a macrolide immunosuppressant; and
   a second film-forming polymer.

As outlined above, the backing layer prevents the active from being released from the therapeutic system at its non-mucosally adhering surface into the saliva, and thus is considered to be able to prevent or reduce unintended delivery via the gastrointestinal route as well as any irritating sensation or discomfort as well as bad taste the macrolide immunosuppressant might cause if present in dissolved form in the oral cavity. Further details on the backing layer will be discussed below in a separate chapter.

The adhesive layer serves to keep the backing layer and the active agent-containing layer adhered to each other. Thus, in certain embodiments, the adhesive layer is adjacent to the active agent-containing layer on its one side and to the backing layer on its other side, i.e., the adhesive layer is “sandwiched” between the backing layer and the active agent-containing layer. The active agent-containing layer, which may be mucoadhesive, may represent the layer that is applied directly to the mucosa.

In certain preferred embodiments, the mucoadhesive layer structure of the transmucosal therapeutic system in accordance with the present invention does not comprise any further layers and consists of the three layers above in view of the ease of manufacture and also in view of keeping the total thickness of the laminate low so as to prevent any patient discomfort. In such a structure, the active agent-containing layer is mucoadhesive and directly contacts the oral mucosa upon administration, i.e., the active agent-containing layer is a mucosa-contacting layer.

In certain other embodiments, the mucoadhesive layer structure may further comprise further layers such as a separate mucosa-contacting layer or cosmetic layers.

Whether provided as an additional layer or not, the mucosa-contacting layer is mucoadhesive and ensures sufficient adhesion to the oral mucosa, preferably for the intended duration of administration and even longer. In contrast to the mucosa-contacting layer, any optional cosmetic layer may provide for a decorative means such as coloring or imprinting, or may simply prevent the patient from touching the backing layer, and is located on top of the mucoadhesive layer structure and is not intended to contact the mucosa.

In view of convenience of manufacture and restricting the overall patch size in terms of surface area, preferably the active agent-containing layer and the adhesive layer, and even more preferably all layers of the mucoadhesive layer structure are coextensive, i.e., identical in shape and size, so that the area of release in such a case corresponds to the area defined by the mucoadhesive layer structure. The mucoadhesive layer structure preferably is large enough to allow a patient convenient manipulating with the fingers without the aid of any specific device such as a tweezer, and a certain minimum size is also required in order to ensure that the laminate does not detach prematurely from the mucosa, and also for being able to include a sufficient amount of active without having to use very thick films. On the other hand, if the laminate is too large, it will be uncomfortable to apply and to wear, leading to low patient compliance. Considering this, in certain embodiments of the invention, the transmucosal therapeutic system has an area of release of at least 0.2 cm², preferably at least 0.5 cm², or has an area of release of less than or equal to 10 cm², preferably less than or equal to 7 cm², or has an area of release of from 0.2 to 10 cm², and more preferably of from 0.5 to 7 cm².

As outlined also above, a mucoadhesive layer structure of a transmucosal therapeutic system consists of one or more thin layers, and thus, in certain embodiments, is in the form of a film. Such a film may have a circular, rectangular or square shape.

The film preferably has a certain degree of thickness, as otherwise it will be difficult to incorporate the required amount of active, and as very thin films are not easy to manufacture, in particular with respect to providing an even thickness. Thus, in certain embodiments, the mucoadhesive layer structure is in the form of a thin film having a total area weight of at least 100 g/m², preferably at least 150 g/m², or more preferably at least 200 g/m². Regarding its thickness, the mucoadhesive layer structure is in the form of a thin film having a total thickness of at least 100 μm, preferably at least 150 μm, and more preferably at least 200 μm. On the other hand, very thick films will be perceived by the patient as disturbing objects in the oral cavity, and thus are disadvantageous in terms of patient compliance. Thus, in certain embodiments, the mucoadhesive layer structure is in the form of a thin film having a total area weight of less than or equal to 450 g/m², preferably less than or equal to 350 g/m², or more preferably less than or equal to 300 g/m². Or, in terms of thickness, the mucoadhesive layer structure is in the form of a thin film having a total thickness of less than 500 μm, preferably less than 400 μm, and more preferably less than 300 μm. In preferred embodiments, the mucoadhesive layer structure is in the form of a thin film having a total area weight of from 100 to 450 g/m², preferably from 150 to 350 g/m², or more preferably from 200 to 300 g/m², or having a total thickness from 100 to 500 μm, preferably from 150 to 400 μm, and more preferably from 200 to 300 μm.

The transmucosal therapeutic system according to the invention is normally stored in a seam-sealed pouch without any further means of protection. However, the mucoadhesive layer structure may also be located on a detachable protective layer (release liner) from which it is removed immediately before application to the mucosa of the patient's oral cavity. Thus, the transmucosal therapeutic system may or may not further comprise a release liner. A transmucosal therapeutic system protected by a release liner is usually also stored in a seam-sealed pouch. The packaging may be child resistant and/or senior friendly.

### Backing Layer

As outlined above in more detail, the transmucosal therapeutic system according to the present invention comprises a mucoadhesive layer structure comprising a backing layer. The backing layer comprises a film-forming polymer, which is ethyl cellulose.

Since the backing layer is used to prevent (at least a substantial amount of) the active to be dissolved into the saliva, the backing layer preferably is water-insoluble, or may be sparingly water-soluble and the time the backing layer takes for dissolution is at least as long the active takes to be delivered through the mucosa, e.g., as long as the active agent-containing layer takes for dissolving. Water-insoluble here means that the backing layer does not dissolve in any aqueous environment, in particular in human saliva. In certain embodiments, the backing layer also does not swell by absorbing water. It is preferred that the backing layer is active agent-free, i.e., in preferred embodiments, the backing layer does not comprise any substantial amounts of any active agent, preferably also not the one contained in the active agent layer.

In terms of surface area size, the backing layer may in particular be of the same size as or larger than the active agent-containing layer. Thus, in certain embodiments, the backing layer and the active agent-containing layer are coextensive in shape and size, while in other embodiments, the backing layer is larger in size than and extends beyond the surface area of the active agent-containing layer. While a mucoadhesive layer structure with the same size of backing and active agent-containing layer is more easy to manufacture since a multi-layer laminate can be diecut to provide the mucoadhesive layer structure, a mucoadhesive layer structure with a backing layer that is larger than the active agent-containing layer is more difficult to manufacture, but also provides the advantage that there is less risk of the active leaking, since the edge of the active-containing layer will also be covered by the backing layer.

With respect to the area weight of the backing layer, a certain thickness is required in order to provide for sufficient protection of active, and in addition, it is also difficult to coat very thin layers in particular with sufficient accuracy. On the other hand, thick layers may not only provoke an uncomfortable feeling in the oral cavity but are also difficult to manufacture. On balance, it is preferred that the backing layer has an area weight of at least 20 g/m², preferably at least 40 g/m² and more preferably of about 50 g/m², or of less than 100 g/m², preferably less than 70 g/m².

As outlined above, the backing layer comprises ethyl cellulose as film-forming polymer which serves as a matrix for providing sufficient cohesive properties of the layer. This film-forming polymer is also referred to as “first” film-forming polymer herein to distinguish between the film-forming polymers used in the other layers of the mucoadhesive layer structure, e.g., the film-forming polymer contained in the active-agent containing layer is referred to as “second” film-forming polymer. The amount of film-forming polymer in the backing layer should be appropriate to provide a layer of sufficient mechanical stability and low water-solubility. Thus, in certain embodiments, the backing layer comprises from 55 to 95 wt-% and preferably about 60 wt-% of the first film-forming polymer. In other embodiments, where the backing layer does not comprise substantial amounts of further excipients, in particular polymers such as the laminating adhesive aid discussed further below, the backing layer may comprise from 70 to 90 wt-%, and preferably about 80 wt-% of ethyl cellulose as the first film-forming polymer. As will be discussed further below, ethyl cellulose is commercially available, e.g., under the brand name Aqualon™ ethylcellulose from Ashland, and in certain embodiments, the ethyl cellulose has
a viscosity as measured as a 5% solution at 25° C. in 80:20 toluene:ethanol by weight in accordance with ASTM D914 of from 30 to 60 mPa·s, more preferably from 40 to 52 mPa·s, and/or
a substitution of ethoxyl groups of 48.0 to 49.5% as measured in accordance with ASTM D914, and/or
a moisture content of 3% by weight or less in accordance with ASTM D914,
   which corresponds to the commercially available grade of ethyl cellulose of N50NF.

The backing layer may comprise, in addition to the (first) film-forming polymer a plasticizer (which is then referred to as “second” plasticizer) and/or a laminating adhesive aid. As will be apparent from the examples, appropriate use of such second plasticizer and/or a laminating adhesive aid will result in an improved adhesion of the laminate.

Thus, in certain embodiments, the backing layer comprises a plasticizer, preferably in an amount of 5 to 20 wt-%, and even more preferably in an amount of about 10 wt-% of the backing layer. Such a plasticizer, if present in the backing layer, is preferably selected from the group consisting of mono-, di-, oligo- and polysaccharides and derivatives thereof, polyethylene glycol, triacetin, triethyl citrate, tributyl citrate, propylene glycol, glycerin, medium chain triglycerides and any mixture thereof. In terms of improved adhesion, the plasticizer, if present in the backing layer, is more preferably glycerin, triacetin or any mixture thereof, and most preferably is triacetin.

As indicated above, in certain embodiments, the backing layer comprises a laminating adhesive aid, preferably in an amount of up to 60 wt-%, or more preferably from 10 to 35 wt-% of the backing layer. Such a laminating adhesive aid, if present in the backing layer, is preferably selected from the group consisting of polyvinylpyrrolidone, polyvinylacetate, vinylpyrrolidone-vinyl acetate copolymers, and any mixture thereof, more preferably is a mixture consisting substantially of polyvinylpyrrolidone and polyvinylacetate or a vinylpyrrolidone-vinyl acetate copolymer, and most preferably is identical to the laminating adhesive present in the adhesive layer. Herein, in certain preferred embodiments, the mixture consisting substantially of polyvinylpyrrolidone and polyvinylacetate comprises and more preferably consists of
from 10 to 30 wt-%, preferably from 15 to 25 wt-% and more preferably about 19 wt-% polyvinylpyrrolidone,
from 70 to 90 wt-%, preferably from 75 to 85 wt-% and more preferably about 80 wt-% polyvinylacetate, and
about 1 wt-% of one or more stabilizers, and preferably about 0.8 wt-% sodium lauryl sulfate and about 0.2 wt-% of silica as stabilizers.

Further preferred embodiments with respect to such a mixture consisting substantially of polyvinylpyrrolidone and polyvinylacetate will be described in further detail below, and are equally preferred for the mixture used here as laminating adhesive aid in order to further enhance the adhesive properties.

Considering the stability of the backing layer with respect to its composition, it is preferable that the backing layer does not comprise any volatile constituents, which bear the risk of evaporating and changing the composition upon storage or of being digested and potentially resulting in health concerns of the patient. Thus, in certain embodiments, the backing layer comprises substantially no (volatile) solvent such as methanol, ethanol, acetone, 1-propanol, 2-propanol, ethyl acetate, hexane, n-heptane, and any mixtures thereof, and in particular, the backing layer comprises less than or equal to 5 wt-%, preferably less than or equal to 3 wt-%, and more preferably less than or equal to 1 wt-% of each of such a solvent.

### Adhesive Layer and Laminating Adhesive

As outlined in more detail above, the transmucosal therapeutic system according to the present invention comprises a mucoadhesive layer structure comprising an adhesive layer. As outlined above, adhesive layers proposed in the past for use in multilayer laminates for mucosal application dissolved too fast in saliva so that they were not suitable for longer administration periods, and also were not suitable for use in systems comprising hydrophobic layers such as water-insoluble backing layers.

The adhesive layer used in the present invention solves this issue by combining low solubility in aqueous media and excellent adhesive properties in particular with respect to adjacent (non-adhesive) layers, which may also be of hydrophobic nature and, e.g., need not comprise a hydrophilic polymer.

Low solubility as used herein means that the adhesive layer withstands dissolution in aqueous media such as saliva for a sufficient duration, e.g., in preferred embodiments, the adhesive layer according to the present invention, provided in the mucoadhesive layer structure between backing layer and active agent-containing layer, will retain integrity if placed in natural or artificial saliva for at least 30 minutes, preferably for at least 60 minutes.

Excellent adhesive properties means that the mucoadhesive layer structure obtained from laminating the adhesive layer between the backing layer and the active agent-containing layer will not show any layer separation. In certain further preferred embodiments, the adhesive layer provides excellent adhesive properties but is still non-tacky to touch, which is of advantage for handling, as, e.g., during manufacture of the mucoadhesive layer structure, the operator does not need to be concerned about unintentional adhering of the adhesive layer to the fingers, hands, or to other surfaces when manipulating the adhesive layer.

In terms of size (i.e., the surface area), and with respect to an individual transmucosal therapeutic system, the adhesive layer may in particular be of the same size as the remaining layers of the mucoadhesive layer structure such as the backing layer and the active agent-containing layer, and preferably is of the same size as at least one of the layers adjacent to the adhesive layer. Thus, in certain embodiments, the adhesive layer and the backing layer and/or the active agent-containing layer are coextensive in shape and size.

With respect to the area weight of the adhesive layer, a certain thickness is required in order to provide for sufficient adhesive properties. On the other hand, the adhesive layer is preferably thin enough so that the total thickness of the transmucosal therapeutic system is such that it is not uncomfortable upon administration in the oral cavity. Thus, in certain embodiments, the adhesive layer has an area weight of at least 30 g/m², preferably at least 50 g/m² and more preferably of about 60 g/m², or of less than 100 g/m² and preferably less than 80 g/m².

As also indicated above, the adhesive layer comprises a laminating adhesive. The laminating adhesive is the main component of the adhesive layer and provides for the adhesive properties. As such, the laminating adhesive is comprised in the adhesive layer in an amount appropriate to provide sufficient adhesiveness, and in certain embodiments, the adhesive layer comprises from 50 to 90 wt-% and preferably from 60 to 85 wt-% of the laminating adhesive.

In certain embodiments, the laminating adhesive is selected from the group consisting of polyvinylpyrrolidone, polyvinylacetate, vinylpyrrolidone-vinyl acetate copolymers, and any mixture thereof, and preferably is a vinylpyrrolidone-vinyl acetate copolymer or a mixture comprising polyvinylpyrrolidone and polyvinylacetate, wherein the mixture even more preferably consists substantially of polyvinylpyrrolidone and polyvinylacetate.

In certain of such embodiments, the vinylpyrrolidone-vinyl acetate copolymer may have a molar ratio of the monomers vinylpyrrolidone and vinyl acetate of about 6:4, a K-Value of from 25 to 31, and/or a molecular weight of from 45,000 to 70,000, and is also referred to as copovidone Ph. Eur. and is available under the brand name Kollidon® VA 64 from BASF.

In the mixture comprising (or consisting of) polyvinylpyrrolidone and polyvinylacetate, the hydrophobic polyvinylacetate is the predominating component that ensures that a sufficient adhesion is achieved also when the adhesive layer is used to laminate relatively hydrophobic layers. Thus, in certain preferred embodiments, the mixture comprises from 70 to 90 wt-%, preferably from 75 to 85 wt-% and more preferably about 80 wt-% polyvinylacetate. The amount of polyvinylpyrrolidone is correspondingly lower, thus, in certain preferred embodiments, the mixture comprises from 10 to 30 wt-%, preferably from 15 to 25 wt-% and more preferably about 19 wt-% polyvinylpyrrolidone.

In certain embodiments, the polyvinylpyrrolidone is selected from soluble polyvinylpyrrolidones and preferably is selected from polyvinylpyrrolidones having a K-Value of from 28 to 32, and/or a molecular weight of from 44,000 to 54,000 and preferably of about 50,000 as measured by SEC using a detection system not requiring reference standards. Such polyvinylpyrrolidones are commercially available, e.g., under the brand name Kollidon® 30 from BASF.

In certain embodiments, the polyvinylacetate has an average molecular weight Mw of from 400,000 to 500,000 and preferably of about 450,000.

In certain embodiments, the mixture may comprise further conventional excipients, and in particular may comprise one or more stabilizers. In certain preferred embodiments, the mixture comprises about 1 wt-% of one or more stabilizers, and preferably comprises about 0.8 wt-% sodium lauryl sulfate and about 0.2 wt-% of silica as stabilizers.

Thus, in an even more preferred embodiment, the mixture comprises
from 10 to 30 wt-%, preferably from 15 to 25 wt-% and more preferably about 19 wt-% polyvinylpyrrolidone,
from 70 to 90 wt-%, preferably from 75 to 85 wt-% and more preferably about 80 wt-% polyvinylacetate, and
about 1 wt-% of one or more stabilizers, and preferably about 0.8 wt-% sodium lauryl sulfate and about 0.2 wt-% of silica as stabilizers.

The average molecular weight of the mixture may be expressed as K-value according to a method described in the monographs “Povidone” and measured in a 1% solution in tetrahydrofuran. In an also preferred embodiment, the mixture has such a K-value of from 60 to 68 and/or a glass transition temperature Tg of about 35° C. Such mixtures are commercially available, e.g., under the brand name Kollidon® SR from BASF which has the further advantage of the mixture being readily at hand and not necessitating a further, time-consuming step of preparing the mixture by combining the individual components.

As also indicated above, the adhesive layer also comprises a plasticizer. The plasticizer serves to increase flexibility and plasticity of the laminating adhesive and is required for achieving appropriate adhesive properties. The plasticizer is therefore also comprised in the adhesive layer in an amount appropriate to provide, together with the laminating adhesive, sufficient adhesiveness. This plasticizer is also referred to as “first” plasticizer herein to distinguish between the plasticizers used in the different layers of the mucoadhesive layer structure, e.g., the plasticizer contained in the backing layer is referred to as “second” plasticizer.

In certain embodiments, the adhesive layer comprises from 10 to 45 wt-% and preferably about 15 wt-% of the first plasticizer.

In accordance with the present invention, the first plasticizer is selected from the group consisting of mono-, di-, oligo- and polysaccharides and derivatives thereof, polyethylene glycol, triacetin, triethyl citrate, tributyl citrate, propylene glycol, glycerin, medium chain triglycerides and any mixture thereof.

As will be also apparent from the examples, the inventors have found out that the adhesive properties can be further enhanced by including a film-forming polymer, however, high amounts of (e.g., hydrophobic) film-forming polymers may result in brittleness of the film, so that the amount is preferably limited. Thus, in certain embodiments, the adhesive layer comprises a film-forming polymer, preferably in an amount of 0 to 10 wt-%, and even more preferably in an amount of about 5 wt-% of the adhesive layer. The film-forming polymer is not particularly limited and can in particular be selected from any of the film-forming polymers listed further below in a separate chapter. In preferred embodiments, the film-forming polymer comprised in the adhesive layer is ethyl cellulose, in particular with characteristics as defined further below. It is further preferred that the film-forming polymer in the adhesive layer is identical to those contained in the layers adjacent to the adhesive layer, i.e. the first film-forming polymer of the backing layer and/or the second film-forming polymer of the active agent-containing layer.

Thus, in certain particularly preferred embodiments, the adhesive layer comprises
from 50 to 90 wt-%, preferably from 60 to 85 wt-% of the laminating adhesive,
from 10 to 45 wt-%, preferably about 15 wt-% of the first plasticizer, and
from 0 to 10 wt-%, preferably about 5 wt-% of the first and/or second film-forming polymer each.

### Active Agent

In accordance with the invention, the transmucosal therapeutic system comprises a therapeutically effective amount of macrolide immunosuppressants as active agent in a mucoadhesive layer structure. In preferred embodiments, substantially all of the active agent is comprised in the active agent-containing layer described further below.

The macrolide immunosuppressant used in the present invention is not particularly limited as long as it is suitable for transmucosal delivery since the inventive adhesive layer allows using a backing layer protecting the active agent-containing layer from being dissolved, bad taste or irritating sensations in the oral cavity caused by the active do not present an obstacle by principle. Since the transmucosal therapeutic system can be designed and adapted to a long period of administration, it is particularly suitable for the macrolide immunosuppressants, which have a relatively high molecular weight and are in general sparingly soluble in saliva and thus require a long time to be delivered.

The macrolide immunosuppressant can be, e.g., selected from the group consisting of tacrolimus, sirolimus, everolimus and pimecrolimus, and more preferably is tacrolimus.

As shown in the examples, the active agent can be incorporated in the active agent-containing layer in a large and in particular therapeutically effective quantity by using a film-forming polymer, which, in particular in preferred embodiments, comprises a mixture of relatively hydrophobic polymers such as ethyl cellulose and hydroxypropyl cellulose, as outlined above. In certain preferred embodiments, the transmucosal therapeutic system comprises a therapeutically effective amount of tacrolimus. In yet further preferred embodiments, the active agent-containing layer comprises at least 0.5 mg/cm² tacrolimus, more preferably at least 0.8 mg/cm² tacrolimus, and most preferably at least 2.0 mg/cm² tacrolimus, and/or comprises less than 6.0 mg/cm², less than 4.0 mg/cm² or less than 2.5 mg/cm² tacrolimus. As is shown in the examples, such a content of tacrolimus in the active agent-containing layer allows for the transmucosal therapeutic system to comprise tacrolimus amounts comparable to those of the commercially available oral capsules. I.e., in certain embodiments, the transmucosal therapeutic system comprises at least 0.1 mg, at least 0.5 mg, at least 1.0 mg or even at least 5.0 mg tacrolimus.

The active agent is preferably incorporated in the film-forming polymer, as will be shown in the chapter concerning the process of manufacture further below, by using an organic solvent such as ethanol, which is capable of dissolving both the (second) film-forming polymer as well as the active agent. Upon removal of the solvent, the active agent remains in the matrix formed by the film-forming polymer in amorphous form (some would refer to such state as the substance being in solution in a polymer matrix, which can also be denominated as “solid solution”), which is advantageously characterized by the absence of any crystalline, but it is understood that also small amounts of crystalline active agent may remain without affecting the performance of the system form of the active agent. The absence of crystalline form can be, e.g., confirmed by X-ray diffraction analysis (XRD), which will reveal the typical 2-Theta (20) peaks in the diffractogram in case the investigated layer comprises active agent in crystalline form. This has been exemplarily shown in the examples for tacrolimus transmucosal therapeutic systems.

Thus, in preferred embodiments of the present invention, the active agent is in amorphous form. In other words, the X-ray diffractogram of the active agent-containing layer is characterized by the absence of reflections of crystalline active agent, as can be determined by comparing with reference layers containing no active agent, or the same active agent but in crystalline form. In certain embodiments, the active agent is present in homogeneously dispersed form, as can be determined by methods known to the skilled person.

In further preferred embodiments, the active agent is tacrolimus in amorphous form, i.e., in other words, the X-ray diffractogram of the active agent-containing layer is characterized by the absence of reflections of crystalline tacrolimus. 2-Theta peaks characteristic for tacrolimus are, e.g., 6.45°, 8.55°, 10.35°, 11.25°, 11.75°, 13.75°, 14.15° and 15.30°. Thus, in further preferred embodiments, the active agent is tacrolimus and the X-ray diffractogram of the active agent-containing layer does not comprise reflections at 2-Theta angles of 6.45°, 8.55°, 10.35°, 11.25°, 11.75°, 13.75°, 14.15° and 15.30°+0.2° degrees. In certain embodiments, the active agent is tacrolimus, and is present in homogeneously dispersed form, as can be determined by methods known to the skilled person.

### Active Agent-Containing Layer

As outlined in more detail above, the transmucosal therapeutic system according to the present invention comprises a mucoadhesive layer structure comprising an active agent-containing layer. The active agent-containing layer comprises a macrolide immunosuppressant as active agent and a film-forming polymer.

As outlined above, the active agent-containing layer preferably is a mucosa-contacting layer and in such specific embodiments needs to provide for sufficient mucoadhesion during the period of administration. As discussed, macrolide immunosuppressants are hydrophobic drugs that need a relatively long time to be delivered transmucosally due to the low solubility. The mucoadhesive properties therefore need to be maintained for a sufficiently long period of time such as 30 minutes, 60 minutes or even longer. The film-forming polymer of the active agent-containing layer is correspondingly chosen to ensure proper mucoadhesive properties, but also in view of potential dissolution or disintegration in the saliva. In certain embodiments, the active agent-containing layer disintegrates over time to release the active, but maintains integrity for the duration of administration, e.g., for at least 30 minutes, 60 minutes or even longer.

As described above in the sections concerning the adhesive layer and the backing layer, these layers may be of the same shape and size as the active agent-containing layer, while in other embodiments, the backing layer is larger in size than and extends the surface area of the active agent-containing layer. In the simplest way and also in accordance with a preferred embodiment, all three layers are coextensive in shape and size.

With respect to the area weight of the active agent-containing layer, a certain minimum thickness is normally required to include a therapeutically effective amount of the macrolide immunosuppressant without the transmucosal therapeutic system becoming too large. Taking into account that the total thickness of the system should be such that it does not cause any discomfort to the patient, it is preferred that the active agent-containing layer has an area weight of at least 100 g/m², preferably at least 130 g/m² and more preferably of about 150 g/m², or of less than 250 g/m², or of less than 200 g/m². Such relatively thick layers can be prepared by repeating the coating and drying of the active agent layer coating composition, e.g., as will be shown in the examples, a 150 g/m² layer can be prepared by coating and drying the coating composition two times in such amount that an area weight of 75 g/m² is achieved each time. In other words, such double (or multi-) layers prepared by repeating the coating and drying of an active agent layer coating composition will be regarded as the active agent-containing layer in the sense of the present invention.

As outlined above, the active agent-containing layer comprises a film-forming polymer which is referred to as “second” film-forming polymer and which serves as a matrix for providing sufficient cohesive properties of the layer. Polymers that can be used as film-forming polymer, and in particular as “second” film-forming polymer are described further below. The amount and type of film-forming polymer in the active agent-containing layer should be appropriate to provide a layer with proper mucoadhesive properties, but also in view of potential dissolution or disintegration in the saliva. Thus, in certain embodiments, the second film-forming polymer is comprised in the active agent-containing layer in a total amount of 70 to 90 wt-% and even more preferably of about 80 wt-% of the active agent-containing layer, and, in particular in case of a mixture of polymers, is comprised in an amount per polymer of 5 to 50 wt-% and preferably 10 to 40 wt-% of the active agent-containing layer. The choice of specific film-forming polymers as second film-forming polymer is outlined further below.

In addition to the second film-forming polymer and the active agent, the active agent-containing layer may comprise further excipients known to the person skilled in the art and as also listed further below. In certain embodiments, the active agent-containing layer comprises one or more further excipients selected from the group consisting of further film-forming polymers, further plasticizers, colorants, antioxidants, taste-masking agents and stabilizers. In certain preferred embodiments, the active agent-containing layer comprises
at least one further film-forming polymer, preferably in an amount of 1 to 6 wt-%, and even more preferably in an amount of 2 to 5 wt-% of the active agent-containing layer each,
a further plasticizer, preferably in an amount of 2 to 10 wt-%, and even more preferably in an amount of 3 to 6 wt-% of the active agent-containing layer, and/or
a colorant, preferably in an amount of 1 to 6 wt-%, and even more preferably in an amount of 2 to 5 wt-% of the active agent-containing layer.

Further preferred specific film-forming polymers, plasticizers, colorants and other excipients are listed below in a separate chapter and can be used appropriately in the active agent-containing layer.

In certain aspects, the present invention is also directed to the active agent-containing layer as such. As outlined above, the inventive active agent-containing layer is advantageous in that it allows incorporation of a sufficient amount of active agent, in particular in a therapeutically effective amount. This applies in particular to tacrolimus. The inventors have been surprisingly successful in being able to show that substantial and therapeutically effective amounts can be incorporated in the active agent-containing layer amorphously, as a solid solution, while at the same time achieving the mechanical and mucoadhesive properties that are required for its use for transmucosal administration and drug delivery. Moreover, such an inventive active agent-containing layer provides for chemical and physical product stability in that it is chemically compatible with the amorphous drug to the extent that a commercially attractive shelf life can be achieved with low chemical degradation of the active ingredient, such degradation generally being a substantial technical risk associated with the amorphous form of an active ingredient. Also, in terms of physical stability, solid solutions of amorphous active ingredients are associated with some increased risk in that many initially promising formulations fail over time as they show re-crystallization of the active ingredient triggered by factors such as temperature fluctuations over storage, mechanical stress, and/or humidity. As demonstrated in the examples, the active release is appropriate for transmucosal delivery in case the active agent is in amorphous form, there is substantially no active agent in crystalline form and no re-crystallization behavior was observed over time for the formulations. Thus, in accordance with one of such aspects, the present invention is also related to an active agent-containing layer for a transmucosal therapeutic system, the active agent-containing layer comprising
i) tacrolimus in amorphous form; and
ii) a film-forming polymer selected from the group consisting of ethyl cellulose, hydroxypropyl cellulose, and any mixtures thereof.
   In certain embodiments of such an aspect, the X-ray diffractogram of the active agent-containing layer is characterized by the absence of reflections of crystalline tacrolimus, and preferably does not comprise reflections at 2-Theta angles of 6.45°, 8.55°, 10.35°, 11.25°, 11.75°, 13.75°, 14.150 and 15.30°±0.2° degrees.

In certain further aspects, the present invention is also directed to the use of such an active agent-containing layer in a transmucosal therapeutic system. I.e., the active agent-containing layer as disclosed herein can also by itself be used as a transmucosal therapeutic system, e.g., in therapeutic applications where the function of the backing layer is not considered to be essential, taking into account in particular administration sites limiting dissolution into the saliva and thus reducing the risk of unintended enteral delivery such as between the gingiva and buccal surface (trapped buccal/gingival site) or sublingual administration (trapped under the tongue). Alternatively, the active agent-containing layer as disclosed herein can be used as part of a transmucosal therapeutic system in combination with an alternative backing layer that may or may not have similar properties and functionality as described for the backing layer of the transmucosal therapeutic system provided herein, but may use a different composition.

In some of the preferred embodiments of an inventive active agent-containing layer and uses thereof, further optional and preferred characteristics concerning the active agent-containing layer and its components such as the active agent, the (second) film-forming polymer, the further film-forming polymer, the plasticizer, the colorant and other excipients are identical to those described further above for the active agent-containing layer as part of the inventive transmucosal therapeutic system.

In certain yet further aspects, the present invention is also directed to a mucoadhesive layer structure for a transmucosal therapeutic system comprising such an active agent-containing layer. In accordance with such an aspect, the present invention is directed to a mucoadhesive layer structure for a transmucosal therapeutic system comprising
A) an active agent-containing layer comprising
   i) tacrolimus in amorphous form; and
   ii) a film-forming polymer selected from the group consisting of ethyl cellulose, hydroxypropyl cellulose, and any mixtures thereof, and
B) an adhesive layer comprising
   a laminating adhesive; and
   a plasticizer.

In certain additional aspects, the present invention is also directed to the use of such a mucoadhesive layer structure in a transmucosal therapeutic system. The surprising and beneficial effects that will be achieved by such an inventive mucoadhesive layer structure and associated uses have been already discussed and the reader is referred to the above sections, in particular to those concerning the active agent and the active agent-containing layer.

In all the above aspects concerning mucoadhesive layer structures for a transmucosal therapeutic system, and uses thereof, further preferred characteristics concerning the mucoadhesive layer structure, the active agent-containing layer and its components such as the active agent, the (second) film-forming polymer, the further film-forming polymer, the plasticizer, the colorant and other excipients are identical to those described further above or below for the mucoadhesive layer structure and in particular the active agent-containing layer as part of the inventive transmucosal therapeutic system.

### Film-Forming Polymers

As outlined in more detail above, the transmucosal therapeutic system according to the present invention comprises a mucoadhesive layer structure comprising a backing layer, an adhesive layer and an active agent-containing layer, and the backing layer and the active agent-containing layer both comprise a film-forming polymer.

The film-forming polymers that can be used in the present invention are not particularly limited and any known polymer with film-forming properties, preferably those that are pharmaceutically acceptable (and, e.g., are approved for pharmaceutical applications), can be employed.

Exemplary film-forming polymers that can be used in the present invention are, e.g., ethyl cellulose (commercially available, e.g., under the brand name Aqualon™ ethylcellulose from Ashland), copolymers based on dimethylaminoethyl methacrylate, butyl methacrylate and methyl methacrylate (commercially available, e.g., under the brand name Eudragit® E100 from Evonik), hydroxypropyl cellulose (commercially available, e.g., under the brand name Klucel™ from Ashland), hydroxypropyl methyl cellulose, polyvinylpyrrolidone, polyethylene oxide, polyethylene glycol, hydroxyethyl cellulose, methacrylic acid-methyl methacrylate copolymer (1:1) (commercially available, e.g., under the brand name Eudragit® L100 from Evonik) in neutralized form, mixed calcium/sodium salt of a methyl vinyl ether and maleic anhydride copolymer (commercially available, e.g., under the brand name Gantrez™ from Ashland), vinylpyrrolidone-vinyl acetate copolymers (commercially available, e.g., under the brand name Kollidon® from BASF) and any mixture thereof.

In certain preferred embodiments, the ethyl cellulose has
a viscosity as measured as a 5% solution at 25° C. in 80:20 toluene:ethanol by weight in accordance with ASTM D914 of from 30 to 60 mPa·s, more preferably from 40 to 52 mPa·s, and/or
a substitution of ethoxyl groups of 48.0 to 49.5% as measured in accordance with ASTM D914, and/or
a moisture content of 3% by weight or less in accordance with ASTM D914.

Such ethyl cellulose is commercially available, e.g., from Ashland Specialty Ingredients and corresponds to the grade ethyl cellulose N50NF.

Hydroxypropyl cellulose is commercially available, e.g., from Ashland under the brand name Klucel™ and is provided in several grades.

The grades differ from each other by molecular weight Mw (as measured by GPC-size exclusion chromatography) and Brookfield viscosity (25° C., LVF, Moisture Free), and are as follows:
The HF grade has a Mw of 1,150,000 and a Brookfield Viscosity of 1,500-3,000 mPa·s (1% in water),
the MF grade has a Mw of 850,000 and a Brookfield Viscosity of 4,000-6,500 mPa·s (2% in water),
the GF grade has a Mw of 370,000 and a Brookfield Viscosity of 150-400 mPa·s (2% in water),
the JF grade has a Mw of 140,000 and a Brookfield Viscosity of 150-400 mPa·s (5% in water),
the LF grade has a Mw of 95,000 and a Brookfield Viscosity of 75-150 mPa·s (5% in water),
the EF grade has a Mw of 80,000 and a Brookfield Viscosity of 300-600 mPa·s (10% in water),
the ELF grade has a Mw of 40,000 and a Brookfield Viscosity of 150-300 mPa·s (10% in water).

Thus, in certain embodiments, the hydroxypropyl cellulose has a molecular weight (as measured by GPC-size exclusion chromatography) of from 30,000 to 1,500,000, and in particular, the hydroxypropyl cellulose has a molecular weight (as measured by GPC-size exclusion chromatography) selected from
between 35,000 and 45,000, in particular 40,000
between 75,000 and 85,000, in particular 80,000
between 90,000 and 100,000, in particular 95,000
between 130,000 and 150,000, in particular 140,000
between 350,000 and 400,000, in particular 370,000,
between 800,000 and 900,000, in particular 850,000, and
between 1,100,000 and 1,200,000, in particular 1,150,000.

In preferred embodiments, the hydroxypropyl cellulose has:
a Mw of about 370,000 and a Brookfield Viscosity of 150-400 mPa·s (2% in water), or
a Mw of about 80,000 and a Brookfield Viscosity of 300-600 mPa·s (10% in water).

In certain embodiments, the mixed calcium/sodium salt of a methyl vinyl ether and maleic anhydride copolymer has a number average molecular weight Mn of about 600,000 to 700,000 and a weight average molecular weight Mw of about 790,000. Such a mixed calcium/sodium salt of a methyl vinyl ether and maleic anhydride copolymer is commercially available, e.g., under the brand name Gantrez™ MS-955 from Ashland.

In certain embodiments, the vinylpyrrolidone-vinyl acetate copolymer may have a molar ratio of the monomers vinylpyrrolidone and vinyl acetate of about 6:4, a K-Value of from 25 to 31, and/or a molecular weight of from 45,000 to 70,000. Such a vinylpyrrolidone-vinyl acetate copolymer is, as also described for the laminating adhesive above, also referred to synonymously as copovidone Ph. Eur. and is available under the brand name Kollidon® VA 64 from BASF.

Where to be used as the first film-forming polymer, i.e., the film-forming polymer comprised in the backing layer, the film-forming polymer needs to be practically water-insoluble to be able to protect the underlying active agent-containing layer. The first film-forming polymer is ethyl cellulose, and preferably is ethyl cellulose of N50NF grade as described above, more preferably used in an amount of from 55 to 95 wt-%, and even more preferably in an amount of about 60 wt-% of the backing layer.

As outlined above, the second film-forming polymer, i.e., the film-forming polymer comprised in the active agent-containing layer, needs to be carefully chosen in order to provide a layer with proper mucoadhesive properties, but also a relatively slow dissolution, erosion or disintegration in the saliva so that the administration period can be long enough to ensure delivery of therapeutically effective amounts of the active. The polymer also needs to enable incorporating sufficient quantities of the active agent. In view of such considerations, the second film-forming polymer is selected from the group consisting of ethyl cellulose, hydroxypropyl cellulose, and any mixtures thereof. Such polymers and mixtures, which are in this context covered by the expression “film-forming polymer”, in particular mixtures of two or more polymers having different degrees of hydrophilicity (or hydrophobicity, respectively), may provide balanced film properties. Ethyl cellulose, hydroxypropyl cellulose, and any mixtures thereof are relatively hydrophobic but still capable of absorbing water and adhere to the mucosa, and it is possible to incorporate large quantities of a hydrophobic active such as macrolide immunosuppressants.

In certain preferred embodiments, where used in the second film-forming polymer, the ethyl cellulose is of N50NF grade as described above and is used preferably in an amount of 20 to 40 wt-% and more preferably 25 to 35 wt-% of the active agent-containing layer, in particular where a mixture of film-forming polymers is used as second film-forming polymer. In certain preferred embodiments, where used in the second film-forming polymer, the hydroxypropyl cellulose preferably is of (i) grade GF as described above, used more preferably in an amount of 5 to 20 wt-% and even more preferably 7 to 15 wt-% of the active agent-containing layer, in particular where a mixture of film-forming polymers is used as second film-forming polymer, or of (ii) grade EF as described above, used more preferably in an amount of 25 to 50 wt-% and even more preferably 30 to 45 wt-% of the active agent-containing layer, in particular where a mixture of film-forming polymers is used as second film-forming polymer. In an even further embodiment, the second film-forming polymer is a mixture of
ethyl cellulose with
   a viscosity as measured as a 5% solution at 25° C. in 80:20 toluene:ethanol by weight in accordance with ASTM D914 of from 30 to 60 mPa·s, more preferably from 40 to 52 mPa·s, and/or
   a substitution of ethoxyl groups of 48.0 to 49.5% as measured in accordance with ASTM D914, and/or
   a moisture content of 3% by weight or less,
preferably in an amount of 20 to 40 wt-% and more preferably 25 to 35 wt-% of the active agent-containing layer,
hydroxypropyl cellulose with
   a molecular weight (as measured by GPC-size exclusion chromatography) of between 350,000 and 400,000, in particular about 370,000, and/or
   a Brookfield Viscosity of 150-400 mPa·s as measured as a 2% solution in water, preferably in an amount of 5 to 20 wt-% and more preferably 7 to 15 wt-% of the active agent-containing layer, and
hydroxypropyl cellulose with
   a molecular weight (as measured by GPC-size exclusion chromatography) of between 75,000 and 85,000, in particular about 80,000, and/or
   a Brookfield Viscosity of 300-600 mPa·s as measured as a 10% solution in water, preferably in an amount of 25 to 50 wt-% and more preferably 30 to 45 wt-% of the active agent-containing layer.

In certain embodiments, the active agent-containing layer comprises a further film-forming polymer, and in such a case, it is preferred that the further film-forming polymer is selected from the group consisting of a mixed calcium/sodium salt of a methyl vinyl ether and maleic anhydride copolymer, which more preferably has a number average molecular weight Mn of about 600,000 to 700,000 and a weight average molecular weight Mw of about 790,000, and a vinylpyrrolidone-vinyl acetate copolymer and any mixture thereof, which more preferably has a molar ratio of the monomers vinylpyrrolidone and vinyl acetate of about 6:4, a K-Value of from 25 to 31, and/or a molecular weight of from 45,000 to 70,000.

In certain embodiments, the adhesive layer also comprises a film-forming polymer, which preferably is identical to the first and/or the second film-forming polymer. In other words, in certain embodiments, the adhesive layer comprises a film-forming polymer selected from the group consisting of ethyl cellulose and hydroxypropyl cellulose. Where used as a film-forming polymer in the adhesive layer, the hydroxypropyl cellulose is preferably of grade EF as described above and the ethyl cellulose is of N50NF grade as described above.

### Plasticizer

As outlined in more detail above, the transmucosal therapeutic system according to the present invention comprises a mucoadhesive layer structure comprising a backing layer, an adhesive layer and an active agent-containing layer, wherein the adhesive layer comprises a plasticizer, and the backing layer and the active agent-containing layer may also comprise a plasticizer.

The plasticizers that can be used in the present invention are not particularly limited and any plasticizer known to the person skilled in the art, preferably those that are pharmaceutically acceptable (and, e.g., are approved for pharmaceutical applications), can be employed.

Exemplary plasticizers that can be used in the present invention are, e.g., mono-, di-, oligo- and polysaccharides and derivatives thereof, polyethylene glycol, triacetin, triethyl citrate, tributyl citrate, propylene glycol, glycerin, medium chain triglycerides and any mixture thereof.

Where used in the backing layer, the plasticizer is referred to as second plasticizer, and can be for example chosen from the plasticizers listed above. In certain preferred embodiments, the second plasticizer is glycerin, triacetin or any mixture thereof, and preferably is triacetin. The second plasticizer is preferably used in an amount of 5 to 20 wt-%, and even more preferably in an amount of about 10 wt-% of the backing layer.

Where used in the adhesive layer, the plasticizer is referred to as first plasticizer, and can be for example chosen from the plasticizers listed above. In certain preferred embodiments, the second plasticizer is glycerin, triacetin or any mixture thereof, and preferably is glycerin. The first plasticizer is preferably used in an amount of from 10 to 45 wt-%, and more preferably of about 15 wt-% of the adhesive layer.

Where used in the active agent-containing layer, the plasticizer can be for example chosen from the plasticizers listed above. In certain preferred embodiments, the plasticizer used in the active agent-containing layer is glycerin, triacetin or any mixture thereof, and more preferably is glycerin, preferably used in an amount of 2 to 10 wt-%, and even more preferably in an amount of 3 to 6 wt-% of the active agent-containing layer.

### Further Excipients

The layers of the transmucosal therapeutic system according to the invention may each comprise further excipients selected from the group consisting of fatty acids, taste-masking agents, sweeteners, flavoring agents, colorants, permeation enhancers, solubilizers, humectants, disintegrants, emulsifiers, antioxidants, stabilizers, buffer reagents and further film-forming polymers.

Such excipients may be present in the layer concerned in an amount of from 0.001 to 15 wt-% of the layer per additive. In a certain embodiment, the total amount of all additives is from 0.001 to 25 wt-% of the layer.

In certain embodiments, the colorant may be selected from the group consisting of titanium dioxide, brilliant blue FCF, indigo carmine, fast green FCF, erythrosine, allura red AC, tartrazine and sunset yellow FCF, curcumin, riboflavin, rivoflavin-5′-phosphate, quinoline yellow, orange yellow S, cochineal, carminic acid, azorubine, carmoisine, amaranth, ponceau 4R, cochineal red A, patent blue V, indigotine, chlorophylls, chlorophyllins, copper complexes of chlorophyll and chlorophyllins, green S, plain caramel, caustic sulphite caramel, ammonia caramel, sulphite ammonia caramel, brilliant black BN, black PN, vegetable carbon, brown HT, carotenes, annatto, bixin, norbixin, paprika extract, capsanthian, capsorubin, lycopene, beta-apo-8′-carotenal, lutein, canthaxanthin, beetroot red, betanin, anthocyanins, calcium carbonate, iron oxides and hydroxides, aluminium, silver, gold and litholrubine BK.

In certain embodiments, the antioxidant may be selected from BHT and vitamin E (α-tocopherol).

In certain embodiments, the stabilizer may be selected from chelating agents such as organic acids selected from mono-, di-, oligo and polycarboxylic acids, for example succinic acid, citric acid, tartaric acid, acrylic acid, benzoic acid, malic acid, maleic acid, oxalic acid, sorbic acid and mixtures thereof.

### Method of Treatment/Medical Use

In accordance with a specific aspect of the present invention, the transmucosal therapeutic system according to the invention is for use in a method of treatment and/or prophylaxis of diseases and medical conditions, preferably in a human patient.

According to a specific aspect, the present invention is also directed to a method of treatment and/or prophylaxis of diseases and medical conditions, wherein the transmucosal therapeutic system according to the invention is administered to a patient, and preferably a human patient.

According to a specific aspect, the present invention is also directed to the use of the inventive transmucosal therapeutic system for the manufacture of a medicament for the treatment and/or prophylaxis of diseases and medical conditions, preferably in a human patient.

The macrolide immunosuppressants used as active agent in the present invention are indicated for the management of organ rejection risk after organ transplant. Thus, in certain embodiments, the treatment and/or prophylaxis of diseases and medical conditions as referred to above is the treatment and/or prophylaxis of organ rejection after organ transplant in a human patient, and more preferably is prophylaxis of organ rejection after allogeneic liver, kidney or heart transplant, in a human patient.

The transmucosal therapeutic system is preferably administered by applying the mucoadhesive layer structure to the mucosa of the oral cavity of a human patient and maintained on the mucosa for a certain period of time (the administration period) and/or until dissolved. In case of insoluble backing or adhesive layers or those that have such a low solubility that it is not completely dissolved during the administration period (which is preferred), these layers need to be removed from the administration site after the administration period or swallowed. In preferred embodiments, the transmucosal therapeutic system is administered by applying the mucoadhesive layer structure to the buccal, sublingual, gingival or palatal mucosa of the oral cavity of a human patient. The administration period is long enough to ensure proper delivery of the active agent and is at least several minutes, such as from 1 to 10 minutes, a little longer, such as 10 to 30 minutes, or even extended such as from 30 minutes to 60 minutes and even longer.

### Process of Manufacture

The invention further relates to a process of manufacture of a transmucosal therapeutic system, and in particular of the inventive transmucosal therapeutic system as outlined above. In accordance with one aspect, the invention also relates to a process of manufacture of an active agent-containing layer as described above.

In such a process of manufacture, a coating composition is prepared for each of a backing layer, an active agent-containing layer and an adhesive layer, by combining all of the layer components with a solvent. The backing layer and active agent-containing layer are then prepared by coating and drying the backing layer coating composition as well as the active agent layer coating composition on a release liner (which can be an intermediate release liner that is later removed), and a bi-layer laminate is obtained by coating and drying the adhesive layer on the backing layer. The thus prepared bi-layer laminate can then be laminated with the active agent-containing layer using, e.g., a conventional lamination station. Lamination preferably does not require the use of a solvent and is thus referred to as “dry bond laminating”. In a yet another preferred embodiment, lamination can also be conducted without application of heat, and thus is advantageous in that the active agent is not subjected to thermal stress.

To summarize, the inventive process of manufacture of a transmucosal therapeutic system comprises the steps of
(a) combining at least the backing layer components as described above and a solvent to obtain a backing layer coating composition, coating the backing layer coating composition onto a release liner and drying the coated backing layer coating composition to form a backing layer,
(b) combining at least the active agent layer components as described above and a solvent to obtain an active agent layer coating composition coating the active agent layer coating composition onto a release liner and drying the coated active agent layer coating composition to form an active agent-containing layer,
(c) combining at least the adhesive layer components as described above and a solvent to obtain an adhesive layer coating composition coating the adhesive layer coating composition onto the backing layer obtained in step (a) and
   drying the coated adhesive layer coating composition to form a bi-layer laminate consisting of an adhesive layer and a backing layer on a release liner, and
(d) laminating the bi-layer laminate obtained in step (c) with the active agent-containing layer obtained in step (b).

In step (b), the coating step is optionally repeated by coating the active agent layer coating composition on the coated active agent layer coating composition before drying, or on the dried active agent-containing layer, to obtain a double-layer active agent-containing layer.

In steps (a) and (b), the release liner in preferred embodiments is an intermediate release liner that can later be removed. In all steps, the drying time is adapted in view of the solvent and matrix polymer used. In step (c), preferably the drying step is conducted only as long as it is necessary to obtain a surface which is dry to touch. Without wishing to be bound by theory, the adhesive layer in such a case may still contain some residual amount of solvent which may be beneficial for the consecutive dry bond laminating step in that a good adhesion can be achieved.

The inventive active agent-containing layer for a transmucosal therapeutic system can be prepared by an analogous process which, however, comprises only step (b) obligatorily. In certain preferred embodiments, in such a process, the active agent is tacrolimus.

In such inventive processes as outlined above, the features of the components used as well as the layers obtained are preferably as outlined above in the corresponding chapters.

The solvent used in each of the steps are not particularly limited, but in view of ease of coating, it is preferred that the solvent is able to dissolve substantially all of the layer components except optional inorganic components such as titanium dioxide, and thus is selected from organic solvents, e.g., selected from ethanol, ethyl acetate, acetone and any mixture thereof. In preferred embodiments, the solvent used in step (a) for the backing layer is selected from organic solvents and preferably is ethanol or a solution of ethanol and acetone. In preferred embodiments, the solvent used in step (b) for the active agent-containing layer is ethanol, which allows dissolving the active and the film-forming polymer (see examples). In view of the need to be able to dissolve the active agent completely, the solvent used in step (b) preferably does not comprise large amounts of water, and more preferably does not comprise any substantial amount of water. In yet preferred embodiments, the solvent used in step (c) for the adhesive layer is selected from organic solvents, preferably from the group consisting of ethanol, ethyl acetate, acetone and any mixtures thereof, and preferably is ethyl acetate, acetone, or a solution of acetone and ethanol.

Finally, in accordance with one aspect, the present invention is also directed to a transmucosal therapeutic system, an active agent-containing layer or a mucoadhesive layer structure that is obtainable by the processes as described above.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Example 1: Coating Compositions

### Backing Layer Coating Compositions

### Formulation

The formulations of the backing layer coating compositions A and B are summarized in Table 1 below. The formulations are based on weight percent, as also indicated in Table 1.

TABLE 1
Backing layer coating compositions

Composition

| | A | B |
|---|---|---|
| | Amount | Amount |
| Substance | [wt-%] | [wt-%] |
| Ethyl cellulose N50NF | 13.49 | 8.99 |
| Kollidon SR | / | 4.5 |
| Glycerol | 1.50 | / |
| Triacetin | / | 1.50 |
| Colorant Brilliant Blue | 0.02 | 0.02 |
| Ethanol | 84.99 | 68.00 |
| Acetone | / | 17.00 |

### Preparation of Coating Composition (Backing Layer)

Backing layer coating compositions A and B were prepared by combining all components of the formulation listed above in Table 1 using standard equipment, e.g., laboratory stirrers and glass beakers, and standard procedures known to the person skilled in the art.

### Adhesive Layer Coating Compositions

### Formulation

The formulations of the adhesive layer coating compositions C and D are summarized in Table 2 below. The formulations are based on weight percent, as also indicated in Table 2.

TABLE 2
Adhesive layer coating compositions

Composition

| | C | D |
|---|---|---|
| | Amount | Amount |
| Substance | [wt-%] | [wt-%] |
| Kollidon VA 64 | 48.0 | / |
| Kollidon SR | / | 36 |
| Glycerol | 12.0 | 6.75 |
| Ethyl cellulose N50NF | / | 2.25 |
| Ethanol | 40.0 | 10 |
| Acetone | / | 45 |

### Preparation of Coating Composition (Adhesive Layer)

Adhesive layer coating compositions C and D were prepared by combining all components of the formulation listed above in Table 2 using standard equipment, e.g., laboratory stirrers and glass beakers, and standard procedures known to the person skilled in the art.

### Active Agent Layer Coating Compositions

### Formulation

The formulations of the active agent layer coating compositions E to H, J and K as well as reference composition I are summarized in Table 3 below. The formulations are based on weight percent, as also indicated in Table 3.

TABLE 3
Active agent layer coating compositions

| | Composition | | | | | | |
|---|---|---|---|---|---|---|---|
| | E | F | G | H | Ref. I | J | K |
| | Amount | Amount | Amount | Amount | Amount | Amount | Amount |
| Substance | [wt-%] | [wt-%] | [wt-%] | [wt-%] | [wt-%] | [wt-%] | [wt-%] |
| Tacrolimus | 1.11 | 1.11 | 1.11 | 1.11 | 1.67 | 2.22 | 2.22 |
| Ethylcellulose | 6.51 | 6.90 | 6.52 | 6.35 | / | 6.14 | 5.97 |
| N50NF | | | | | | | |
| Soluplus* | / | / | / | / | 4.73 | / | / |
| Hydroxypropyl- | 8.07 | 7.31 | 7.31 | 7.90 | 6.38 | 6.88 | 7.43 |
| cellulose EF | | | | | | | |
| Hydroxypropyl- | 1.89 | 2.27 | 2.65 | 1.72 | / | 2.49 | 1.61 |
| cellulose GF | | | | | | | |
| NaCMC 7LP EP** | / | / | / | / | 4.26 | / | / |
| Kollidon VA 64 | / | / | / | 0.50 | / | / | 0.50 |
| Gantrez MS 955 | 0.76 | 0.75 | 0.75 | 0.76 | / | 0.71 | 0.71 |
| Glycerol | 0.95 | 0.94 | 0.94 | 0.95 | 2.45 | 0.89 | 0.89 |
| Titan dioxide | 0.72 | 0.72 | 0.72 | 0.72 | 0.51 | 0.67 | 0.68 |
| Ethanol | 80.00 | 80.00 | 80.00 | 80.00 | / | 80.00 | 80.00 |
| Water | / | / | / | / | 80.00 | / | / |

*Soluplus ® is a polyethylene glycol-polyvinyl acetate- and polyvinylcaprolactame-based graft copolymer commercially available from BASF.
**NaCMC 7LP EP is a sodium carboxymethyl cellulose with a degree of substitution of 0.7, a weight average molecular weight of 90,500 and a viscosity of 25-50 mPa · s.

### Preparation of Coating Composition (Active Agent-Containing Layer)

Active agent layer coating compositions E to K were prepared by combining all components of the formulation listed above in Table 3 using standard equipment, e.g., laboratory stirrers and glass beakers, and standard procedures known to the person skilled in the art. The active agent tacrolimus was dissolved in coating compositions E to H, J and K, while for reference composition I, tacrolimus could not be dissolved due to its low solubility in water, and thus is present in suspended state.

### Example 2: Manufacturing of Transmucosal Therapeutic Systems

Transmucosal therapeutic systems were manufactured as described in the following. Coating and drying of the coating compositions as well as lamination was carried out using standard equipment such as coating box or coating knife for casting, drying cabinets for drying, and conventional laminating stations for lamination, by standard procedures known to the person skilled in the art. The composition and area weight of each of the layers as well as the amount of active agent of the individual transmucosal therapeutic systems 2E to 2K are indicated in Table 4 further below.

### Preparation of Bi-Layer Laminate

Bi-layer laminates were prepared by coating and drying the backing layer coating composition B obtained in Example 1 on a release liner to form the backing layer, and by coating and drying the adhesive layer coating composition D obtained in Example 1 on top of the dried backing layer to obtain an adhesive layer/backing layer bi-layer laminate on a release liner. The coating thicknesses were adjusted to afford an area weight of 50 g/m² for the backing layer and 60 g/m² for the adhesive layer.

### Preparation of Single- and Double-Layer Active Agent-Containing Layers

A single-layer active agent-containing layer was prepared by coating and drying the reference coating composition I obtained in Example 1 on a release liner to obtain reference layer I. The coating thickness was adjusted to afford an area weight of 100 g/m².

Double-layer active agent-containing layers were prepared by coating and drying the active agent layer coating compositions E to H, J and K obtained in Example 1 on a release liner to form single-layer active agent-containing layers, and by repeating the coating and drying using the same active agent layer coating composition on top of the single-layer active agent-containing layer to obtain double-layer active agent-containing layers E to H, J and K. The coating thicknesses were adjusted to afford a total area weight of 150 g/m² (2 layers of 75 g/m² each). As mentioned above, such double layers are also covered by the expression “active agent-containing layer”.

### Preparation of Transmucosal Therapeutic Systems 2E to 2K

Transmucosal therapeutic systems 2E to 2K were prepared by laminating the adhesive layer/backing layer bi-layer laminate obtained with each of the double-layer active agent-containing layers E to K (with the laminate and layers E to K arranged so that the adhesive layer faces the active agent-containing layer), removing the release liner covering the backing layer, and by punching out individual transmucosal therapeutic systems 6 cm² in size.

The transmucosal therapeutic systems were shown to provide sufficient mucoadhesion by preliminary in vivo investigations using placebo layers containing no active agent.

TABLE 4
Transmucosal therapeutic systems

| Substance | 2E | 2F | 2G | 2H | Ref. 2I | 2J | 2K |
|---|---|---|---|---|---|---|---|
| | Backing layer/Amount [wt-%] | | | | | | |
| Ethyl cellulose | 59.90 | | | | | | |
| N50NF | | | | | | | |
| Kollidon SR | 30.00 | | | | | | |
| Triacetin | 10.00 | | | | | | |
| Colorant Brilliant | 0.10 | | | | | | |
| Blue | | | | | | | |
| Area weight (dry) | 50 g/m² | | | | | | |
| | Adhesive layer/Amount [wt-%] | | | | | | |
| Kollidon SR | 80 | | | | | | |
| Glycerol | 15 | | | | | | |
| Ethylcellulose | 5 | | | | | | |
| N50NF | | | | | | | |

| | |
|---|---|
| Area weight (dry) | 60 g/m² |

| | Active agent layer/Amount [wt-%] | | | | | | |
|---|---|---|---|---|---|---|---|
| Substance | E | F | G | H | Ref. I | J | K |
| Tacrolimus | 5.55 | 5.55 | 5.55 | 5.55 | 8.33 | 11.11 | 11.11 |
| Ethylcellulose | 32.57 | 34.49 | 32.59 | 31.74 | / | 30.68 | 29.84 |
| N50NF | | | | | | | |
| Soluplus | / | / | / | / | 23.64 | / | / |
| Hydroxypropyl- | 40.34 | 36.56 | 36.55 | 39.51 | 31.92 | 34.42 | 37.14 |
| cellulose EF | | | | | | | |
| Hydroxypropyl- | 9.44 | 11.33 | 13.23 | 8.60 | / | 12.44 | 8.05 |
| cellulose GF | | | | | | | |
| NaCMC 7LP EP | / | / | / | / | 21.29 | / | / |
| Kollidon VA 64 | / | / | / | 2.50 | / | / | 2.50 |
| Gantrez MS 955 | 3.78 | 3.78 | 3.77 | 3.78 | / | 3.54 | 3.54 |
| Glycerol | 4.73 | 4.71 | 4.72 | 4.73 | 12.27 | 4.44 | 4.44 |
| Titan dioxide | 3.59 | 3.59 | 3.59 | 3.59 | 2.55 | 3.37 | 3.38 |

| Area weight (dry) | 150 g/m²* | 100 g/m² | 150 g/m²* |
|---|---|---|---|
| Tacrolimus | 5 mg/6 cm² | | 10 mg/6 cm² |
| [mg/system size] | | | |

*double-layer

### Example 3: Presence of Crystalline Tacrolimus

Active agent-containing layers E and J as well as reference layer I were examined for the presence of crystalline tacrolimus immediately after manufacturing. In addition, active agent-containing layer K was also examined after 2.5 months at 25° C. The examination was conducted using XRD using crystalline tacrolimus and placebo layers as reference. Placebo layers were prepared in the same way as the active agent-containing layers E, J and K as well as reference layer I as outlined in Examples 1 and 2, with the exception that the formulation of coating compositions E, J and K as well as of reference composition I was changed by omitting the active agent tacrolimus and linearly extrapolating the amount of the remaining constituents equally to 100%.

The films to be measured were fixed on a conventional zero-background film for reflection measurements. X-ray diffractograms XRD were recorded in a 2Θ-range of 2° to 40° for the active agent-containing layers E, I, J, and K as well as Placebo Layers E, I, J, K, and an X-ray powder diffractogram XRPD was obtained in the same 20-range of 2° to 40° for pure crystalline tacrolimus. The integration time (time per step) was varied to improve the detection limits. The measurement parameters are summarized in Table 5 below.

TABLE 5

| Parameter | Value |
|---|---|
| Instrument / Manufacturer | CubiX³ Pharma / Panalytical |
| X-ray tube | Cu LFF HR |
| geometry | reflection |
| X-ray mirror | none |
| Soller slits | 0.02 rad |
| Detector | X'Celerator |
| active length | 2.55 |
| Divergence slit | fixed |
| Divergence slit size | ⅛° |
| X-ray excitation | 40 mA, 40 kV |
| 2Θ-range | 2°-40° |
| measuring mode | continuous |
| time per step | 200 s / 400 s |
| step size | 0.02° |
| Rotation | 1 Rev/s |

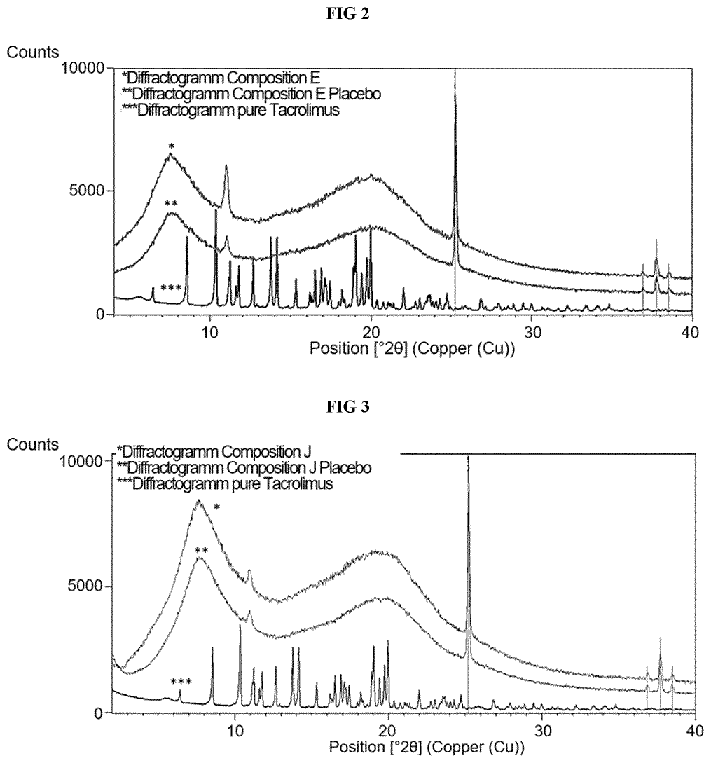

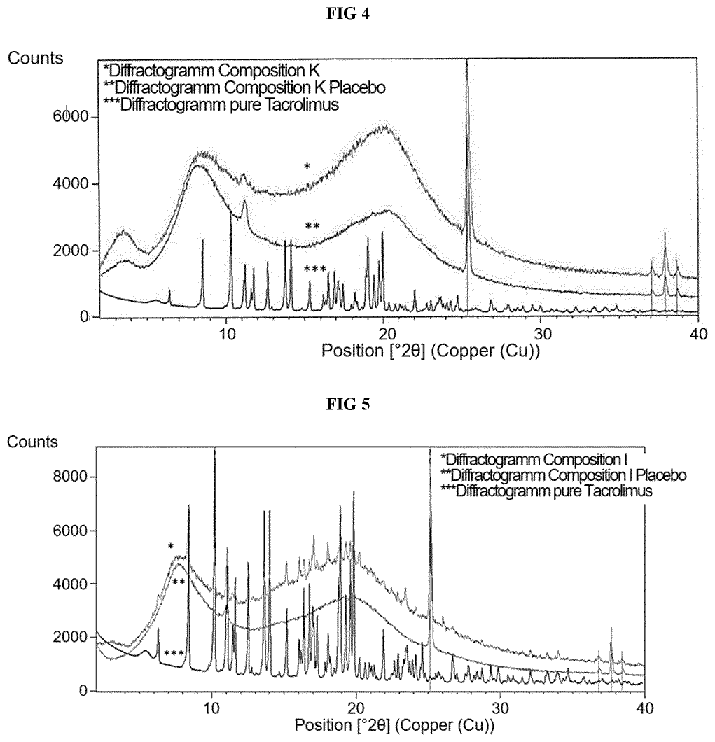

The diffractograms recorded are shown in FIG. 2 (Layer E, Placebo Layer E and pure tacrolimus), FIG. 3 (Layer J, Placebo Layer J and pure tacrolimus), FIG. 4 (Layer K, Placebo Layer K and pure tacrolimus), and FIG. 5 (Ref. Layer I, Placebo Layer I and pure tacrolimus).

As can be seen in FIG. 5, the diffractogram of Reference Layer I shows peaks also present in the diffractogram of pure, crystalline tacrolimus, while in Placebo Layer I the peaks are absent. This confirms that tacrolimus is present in crystalline form in reference layer I and in suspended form in coating composition I, and that the X-Ray measurement employed is appropriate for investigating the presence of crystalline tacrolimus in the active agent-containing layers.

On the other hand, as is apparent from FIG. 2 to 4, the peaks characteristic for tacrolimus present in the diffractogram of pure, crystalline tacrolimus are absent in the diffractograms for the active agent-containing layers E, J and K as well as the corresponding Placebo Layers. This confirms that these layers do not comprise crystalline tacrolimus, and that tacrolimus is present only in amorphous form.

In addition, layer K as well as Placebo Layer K were stored for 2.5 months under controlled conditions at 25° C. and at 60% relative humidity, in contrast to layers E and J, for which measurements were performed directly after manufacturing. This shows that the active agent-containing layers are also stable against recrystallisation upon storage.

### Example 4: In Vitro Release Experiments

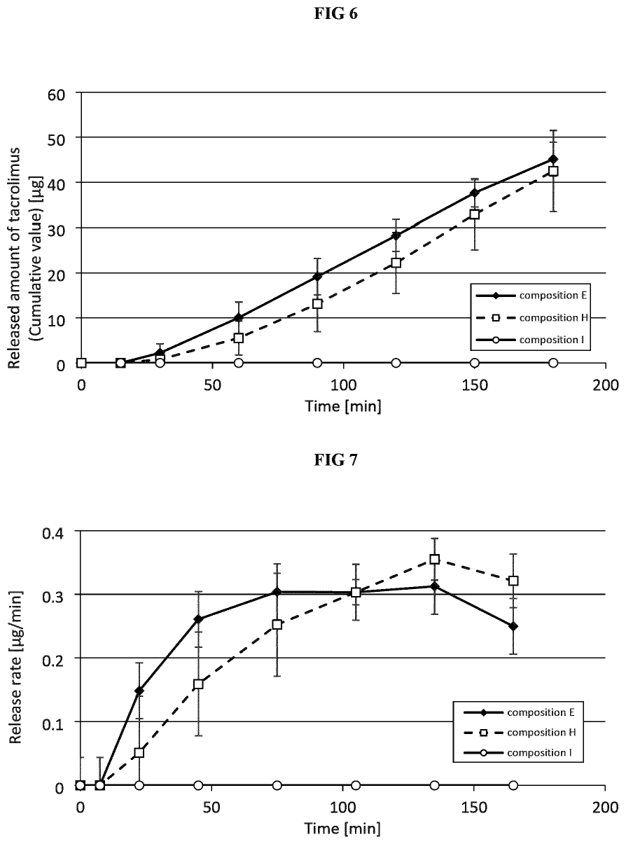

The released amount of tacrolimus and the corresponding release rate was determined for transmucosal therapeutic systems E, H and I. The determination of the release rate was conducted using standard Franz Cells (with a volume of 24 mL). Donor and acceptor compartment were separated using a membrane (Spectra Por®7 dialysis membrane 25 kD). Diecuts with an area of 0.525 cm² were punched from each of the transmucosal therapeutic systems, and applied to the membrane in the donor compartment, which was filled with 600 μl of acetate buffer pH 4.0. The acceptor compartment was filled with 24 mL of acetate buffer pH 4. The experiment was conducted for 180 min. During the experiment the temperature of the Franz cells were kept constant at 32° C.±0.5° C. using a water bath, and the acceptor medium was stirred continuously at 350 rpm. A complete exchange of the acceptor medium was conducted at 15, 30, 60, 90, 120, 150 and 180 minutes. The released amount of tacrolimus in the exchanged acceptor medium was measured by HPLC and the corresponding release rate calculated. No solubilizers such as SDS were used to prevent the occurrence of false positive results. The results are shown in Table 6 and 7 as well as in FIGS. 6 and 7, respectively.

TABLE 6

| | Released amount of tacrolimus | | |
|---|---|---|---|
| Elapsed | (cumulative values) [μg] | | |
| time [min] | Ex. 2E | Ex. 2H | Ref. Ex. 2I |
| 15 | 0.00 | 0.00 | 0.00 |
| 30 | 2.23 | 0.77 | 0.00 |
| 60 | 10.05 | 5.54 | 0.00 |
| 90 | 19.17 | 13.11 | 0.00 |
| 120 | 28.26 | 22.21 | 0.00 |
| 150 | 37.64 | 32.86 | 0.00 |
| 180 | 45.13 | 42.49 | 0.00 |

TABLE 7

| | Tacrolimus release rate | | |
|---|---|---|---|
| Elapsed | [μg / min] | | |
| time [min] | Ex. 2E | Ex. 2H | Ref. Ex. 2I |
| 15 | 0.00 | 0.00 | 0.00 |
| 30 | 0.15 | 0.05 | 0.00 |
| 60 | 0.26 | 0.16 | 0.00 |
| 90 | 0.30 | 0.25 | 0.00 |
| 120 | 0.30 | 0.30 | 0.00 |
| 150 | 0.31 | 0.35 | 0.00 |
| 180 | 0.25 | 0.32 | 0.00 |

For the transmucosal therapeutic system of reference Example 2I, in which tacrolimus is present in crystalline (and dispersed) form, no tacrolimus could be detected in the acceptor medium at any time. This is assumed to be due to the poor water solubility of tacrolimus resulting in no tacrolimus being released from the transmucosal therapeutic system of reference Example 2I, (due to the absence of a solubilizer), so that no tacrolimus could permeate the membrane.

On the other hand, as is apparent from tables 5 and 6 above as well as FIGS. 5 and 6, tacrolimus could indeed be shown to be present in the acceptor medium for the transmucosal therapeutic systems of Examples 2E and 2H, confirming that tacrolimus could permeate through the membrane. These results support that the inventive transmucosal therapeutic systems are capable of releasing tacrolimus transmucosally under in vivo conditions.

1. Transmucosal therapeutic system for the transmucosal administration of an active agent comprising
a mucoadhesive layer structure comprising
A) a backing layer comprising a first film-forming polymer,
B) an adhesive layer comprising
a laminating adhesive; and
a first plasticizer, and
C) an active agent-containing layer comprising
a macrolide immunosuppressant; and
a second film-forming polymer,
wherein
the first film-forming polymer is ethyl cellulose,
the macrolide immunosuppressant is selected from the group consisting of tacrolimus, sirolimus, everolimus and pimecrolimus,
and
the second film-forming polymer is selected from the group consisting of ethyl cellulose, hydroxypropyl cellulose, and any mixtures thereof.

2. Transmucosal therapeutic system according to claim 1, wherein
the macrolide immunosuppressant is tacrolimus and preferably is tacrolimus in amorphous form.

3. Transmucosal therapeutic system according to claim 1 or 2, wherein
the transmucosal therapeutic system comprises a therapeutically effective amount of tacrolimus, and
the active agent-containing layer preferably comprises at least 0.5 mg/cm² tacrolimus, more preferably at least 0.8 mg/cm² tacrolimus, and most preferably at least 2.0 mg/cm² tacrolimus, and/or comprises less than 6.0 mg/cm², less than 4.0 mg/cm² or less than 2.5 mg/cm² tacrolimus.

4. Transmucosal therapeutic system according to any one of claims 1 to 3, wherein
the second film-forming polymer is a mixture of
ethyl cellulose with
a viscosity as measured as a 5% solution at 25° C. in 80:20 toluene:ethanol by weight in accordance with ASTM D914 of from 30 to 60 mPa·s, more preferably from 40 to 52 mPa·s, and/or
a substitution of ethoxyl groups of 48.0 to 49.5% as measured in accordance with ASTM D914, and/or
a moisture content of 3% by weight or less,
preferably in an amount of 20 to 40 wt-% and more preferably 25 to 35 wt-% of the active agent-containing layer,
hydroxypropyl cellulose with
a molecular weight (as measured by GPC-size exclusion chromatography) of between 350,000 and 400,000, in particular about 370,000, and/or
a Brookfield Viscosity of 150-400 mPa·s as measured as a 2% solution in water,
preferably in an amount of 5 to 20 wt-% and more preferably 7 to 15 wt-% of the active agent-containing layer, and
hydroxypropyl cellulose with
a molecular weight (as measured by GPC-size exclusion chromatography) of between 75,000 and 85,000, in particular about 80,000, and/or
a Brookfield Viscosity of 300-600 mPa·s as measured as a 10% solution in water,
preferably in an amount of 25 to 50 wt-% and more preferably 30 to 45 wt-% of the active agent-containing layer.

5. Transmucosal therapeutic system according to any one of claims 1 to 4, wherein
the first film-forming polymer is ethyl cellulose with
a viscosity as measured as a 5% solution at 25° C. in 80:20 toluene:ethanol by weight in accordance with ASTM D914 of from 30 to 60 mPa·s, more preferably from 40 to 52 mPa·s, and/or
a substitution of ethoxyl groups of 48.0 to 49.5%, and/or
a moisture content of 3% by weight or less,
preferably in an amount of from 55 to 95 wt-%, and even more preferably in an amount of about 60 wt-% of the backing layer.

6. Transmucosal therapeutic system according to any one of claims 1 to 5, wherein
the backing layer is water-insoluble and preferably further comprises a second plasticizer, preferably in an amount of 5 to 20 wt-%, and even more preferably in an amount of about 10 wt-% of the backing layer, wherein the second plasticizer is preferably selected from the group consisting of mono-, di-, oligo- and polysaccharides and derivatives thereof, polyethylene glycol, triacetin, triethyl citrate, tributyl citrate, propylene glycol, glycerin, medium chain triglycerides and any mixture thereof, and more preferably is triacetin, and/or a laminating adhesive aid, preferably in an amount of up to 60 wt-%, or more preferably from 10 to 35 wt-% of the backing layer, wherein preferably the laminating adhesive aid is selected from the group consisting of polyvinylpyrrolidone, polyvinylacetate, vinylpyrrolidone-vinyl acetate copolymers, and any mixture thereof, more preferably is a mixture consisting substantially of polyvinylpyrrolidone and polyvinylacetate or a vinylpyrrolidone-vinyl acetate copolymer, and most preferably is identical to the laminating adhesive.

7. Transmucosal therapeutic system according to any one of claims 1 to 6, wherein
the adhesive layer is adjacent to the active agent-containing layer on its one side and to the backing layer on its other side.

8. Transmucosal therapeutic system according to any one of claims 1 to 7, wherein the first plasticizer is selected from the group consisting of mono-, di-, oligo- and polysaccharides and derivatives thereof, polyethylene glycol, triacetin, triethyl citrate, tributyl citrate, propylene glycol, glycerin, medium chain triglycerides and any mixture thereof, and preferably is glycerin or triacetin and more preferably is glycerin.

9. Transmucosal therapeutic system according to any one of claims 1 to 8, wherein
the laminating adhesive is selected from the group consisting of polyvinylpyrrolidone, polyvinylacetate, vinylpyrrolidone-vinyl acetate copolymers, and any mixture thereof, and preferably is a mixture consisting substantially of polyvinylpyrrolidone and polyvinylacetate or a vinylpyrrolidone-vinyl acetate copolymer, wherein more preferably
said mixture comprises
from 10 to 30 wt-%, preferably from 15 to 25 wt-% and more preferably about 19 wt-% polyvinylpyrrolidone,
from 70 to 90 wt-%, preferably from 75 to 85 wt-% and more preferably about 80 wt-% polyvinylacetate, and
about 1 wt-% of one or more stabilizers, and preferably about 0.8 wt-% sodium lauryl sulfate and about 0.2 wt-% of silica as stabilizers, and/or
said mixture has a K-value of from 60 to 68 and/or a glass transition temperature Tg of about 35° C., and/or
the polyvinylpyrrolidone is selected from soluble polyvinylpyrrolidones and preferably is selected from polyvinylpyrrolidones having a K-Value of from 28 to 32, and/or a molecular weight of from 44,000 to 54,000, and/or
the polyvinylacetate has a molecular weight of from 400,000 to 500,000 and preferably of about 450,000, and/or
the vinylpyrrolidone-vinyl acetate copolymer has a molar ratio of the monomers vinylpyrrolidone and vinyl acetate of about 6:4, a K-Value of from 25 to 31, and/or a molecular weight of from 45,000 to 70,000.

10. Transmucosal therapeutic system according to any one of claims 1 to 9, wherein
the adhesive layer further comprises a film-forming polymer which is preferably identical to the first and/or the second film-forming polymer, and more preferably
the adhesive layer comprises
from 50 to 90 wt-%, preferably from 60 to 85 wt-% of the laminating adhesive,
from 10 to 45 wt-%, preferably about 15 wt-% of the first plasticizer, and
from 0 to 10 wt-%, preferably about 5 wt-% of the first and/or second film-forming polymer each.

11. Transmucosal therapeutic system according to any one of claims 1 to 10, wherein
the active agent-containing layer is a mucosa-contacting layer and preferably further comprises one or more further excipients selected from the group consisting of further film-forming polymers, further plasticizers, colorants, antioxidants, taste-masking agents and stabilizers, and more preferably comprises
at least one further film-forming polymer, preferably in an amount of 1 to 6 wt-%, and even more preferably in an amount of 2 to 5 wt-% of the active agent-containing layer each, wherein the further film-forming polymer is selected from the group consisting of a mixed calcium/sodium salt of a methyl vinyl ether and maleic anhydride copolymer and a vinylpyrrolidone-vinyl acetate copolymer and any mixture thereof,
a further plasticizer, preferably in an amount of 2 to 10 wt-%, and even more preferably in an amount of 3 to 6 wt-% of the active agent-containing layer, wherein the further plasticizer is selected from the group consisting of mono-, di-, oligo- and polysaccharides and derivatives thereof, polyethylene glycol, triacetin, triethyl citrate, tributyl citrate, propylene glycol, glycerin, medium chain triglycerides and any mixture thereof, and preferably is glycerin, and/or a colorant, preferably in an amount of 1 to 6 wt-%, and even more preferably in an amount of 2 to 5 wt-% of the active agent-containing layer, wherein the colorant is selected from the group consisting of titanium dioxide, brilliant blue FCF, indigo carmine, fast green FCF, erythrosine, allura red AC, tartrazine and sunset yellow FCF, curcumin, riboflavin, rivoflavin-5′-phosphate, quinoline yellow, orange yellow S, cochineal, carminic acid, azorubine, carmoisine, amaranth, ponceau 4R, cochineal red A, patent blue V, indigotine, chlorophylls, chlorophyllins, copper complexes of chlorophyll and chlorophyllins, green S, plain caramel, caustic sulphite caramel, ammonia caramel, sulphite ammonia caramel, brilliant black BN, black PN, vegetable carbon, brown HT, carotenes, annatto, bixin, norbixin, paprika extract, capsanthian, capsorubin, lycopene, beta-apo-8′-carotenal, lutein, canthaxanthin, beetroot red, betanin, anthocyanins, calcium carbonate, iron oxides and hydroxides, aluminium, silver, gold and litholrubine BK.

12. Transmucosal therapeutic system according to any one of claims 1 to 11, wherein
the backing layer has an area weight of at least 20 g/m², preferably at least 40 g/m² and more preferably of about 50 g/m², or of less than 100 g/m², preferably less than 70 g/m², and/or
the adhesive layer has an area weight of at least 30 g/m², preferably at least 50 g/m² and more preferably of about 60 g/m², or of less than 100 g/m², preferably less than 80 g/m², and/or
the active agent-containing layer has an area weight of at least 100 g/m², preferably at least 130 g/m² and more preferably of about 150 g/m², or of less than 250 g/m², or of less than 200 g/m².

13. Process of manufacture of a transmucosal therapeutic system comprising the steps of:
(a) combining at least
a first film-forming polymer and
a solvent
to obtain a backing layer coating composition,
coating the backing layer coating composition onto a release liner and
drying the coated backing layer coating composition to form a backing layer,
(b) combining at least
an active agent,
a second film-forming polymer, and
a solvent
to obtain an active agent layer coating composition,
coating the active agent layer coating composition onto a release liner and
drying the coated active agent layer coating composition to form a single-layer active agent-containing layer, optionally repeating the coating step by coating the active agent layer coating composition on the coated active agent layer coating composition before drying, or on the dried active agent-containing layer to obtain a double-layer active agent-containing layer,
(c) combining at least
a laminating adhesive, and
a first plasticizer, and
a solvent
to obtain an adhesive layer coating composition,
coating the adhesive layer coating composition onto the backing layer obtained in step (a), and
drying the coated adhesive layer coating composition to form a bi-layer laminate consisting of an adhesive layer and a backing layer on the release liner, and
(d) laminating the bi-layer laminate obtained in step (c) with the single- or double-layer active agent-containing layer obtained in step (b),
wherein
the first film-forming polymer is ethyl cellulose,
the macrolide immunosuppressant is selected from the group consisting of tacrolimus, sirolimus, everolimus and pimecrolimus,
and
the second film-forming polymer is selected from the group consisting of ethyl cellulose, hydroxypropyl cellulose, and any mixtures thereof.

14. Transmucosal therapeutic system according to any one of claims 1 to 12 for use in a method of treatment, preferably for the treatment and/or prophylaxis of organ rejection after organ transplant in a human patient, and more preferably for the prophylaxis of organ rejection after allogeneic liver, kidney, or heart transplant, in a human patient.

15. An active agent-containing layer for a transmucosal therapeutic system, the active agent-containing layer comprising
i) tacrolimus in amorphous form; and
ii) a film-forming polymer selected from the group consisting of ethyl cellulose, hydroxypropyl cellulose, and any mixtures thereof.

16. An active agent-containing layer according to claim 15, wherein
the X-ray diffractogram of the active agent-containing layer is characterized by the absence of reflections of crystalline tacrolimus, and preferably does not comprise reflections at 2-Theta angles of 6.45°, 8.55°, 10.35°, 11.25°, 11.75°, 13.75°, 14.15° and 15.30°+0.2° degrees.

17. A mucoadhesive layer structure for a transmucosal therapeutic system comprising
A) the active agent-containing layer according to claim 16 and
B) an adhesive layer comprising
a laminating adhesive; and
a plasticizer.

18. Transmucosal therapeutic system for the transmucosal administration of an active agent comprising
a mucoadhesive layer structure consisting of
A) a backing layer comprising
from 55 to 95 wt-% and preferably about 60 wt-% of ethyl cellulose with a viscosity as measured as a 5% solution at 25° C. in 80:20 toluene:ethanol by weight in accordance with ASTM D914 of from 30 to 60 mPa·s, more preferably from 40 to 52 mPa·s, and a substitution of ethoxyl groups of 48.0 to 49.5% as measured in accordance with ASTM D914,
from 5 to 20 wt-% and preferably about 10 wt-% triacetin, and
from 10 to 35 wt-% and preferably about 30 wt-% of a mixture consisting of
about 19 wt-% polyvinylpyrrolidone,
about 80 wt-% polyvinylacetate,
about 0.8 wt-% sodium lauryl sulfate and
about 0.2 wt-% of silica,
B) an adhesive layer adjacent to the active agent-containing layer on its one side and to the backing layer on its other side, comprising
from 70 to 90 wt-% and preferably about 80 wt-% of a mixture consisting of
about 19 wt-% polyvinylpyrrolidone,
about 80 wt-% polyvinylacetate,
about 0.8 wt-% sodium lauryl sulfate and
about 0.2 wt-% of silica,
from 10 to 30 wt-%, preferably about 15% of glycerin, and
from 0 to 10 wt-%, preferably about 5 wt-% of ethyl cellulose with a viscosity as measured as a 5% solution at 25° C. in 80:20 toluene:ethanol by weight in accordance with ASTM D914 of from 30 to 60 mPa·s, more preferably from 40 to 52 mPa·s, and a substitution of ethoxyl groups of 48.0 to 49.5% as measured in accordance with ASTM D914,
C) an active agent-containing layer comprising
at least 0.8 mg/cm² of tacrolimus in amorphous form;
ethyl cellulose with a viscosity as measured as a 5% solution at 25° C. in 80:20 toluene:ethanol by weight in accordance with ASTM D914 of from 30 to 60 mPa·s, more preferably from 40 to 52 mPa·s, and a substitution of ethoxyl groups of 48.0 to 49.5% as measured in accordance with ASTM D914, in an amount of 25 to 35 wt-% of the active agent-containing layer,
hydroxypropyl cellulose with a molecular weight (as measured by GPC-size exclusion chromatography) of about 370,000, and a Brookfield Viscosity of 150-400 mPa·s as measured as a 2% solution in water, in an amount of 7 to 15 wt-% of the active agent-containing layer, and
hydroxypropyl cellulose with a molecular weight (as measured by GPC-size exclusion chromatography) of about 80,000, and a Brookfield Viscosity of 300-600 mPa·s as measured as a 10% solution in water, in an amount of 30 to 45 wt-% of the active agent-containing layer,
a mixed calcium/sodium salt of a methyl vinyl ether and maleic anhydride copolymer as a further film-forming polymer in an amount of 2 to 5 wt-% of the active agent-containing layer,
optionally a vinylpyrrolidone-vinyl acetate copolymer in an amount of 1 to 4 wt-% of the active agent-containing layer, and
glycerin in an amount of 3 to 6 wt-% of the active agent-containing layer.
